(19) United States
(12) Patent Application Publication
(10) Pub. No.: US 2026/0137513 A1
(43) Pub. Date: May 21, 2026

(54) DOCKING STATION WITH IMPROVED FLEXIBILITY

(71) Applicant: Edwards Lifesciences Corporation, Irvine, CA (US)

(72) Inventors: Michael G. Valdez, Riverside, CA (US); Amirsepehr Azimian, Costa Mesa, CA (US); Omar Fawzi Azanki, Coto de Caza, CA (US); Anne Bernadette Aragon Alcasid, Costa Mesa, CA (US); David Lyle Leibowitz, Capistrano Beach, CA (US); Tiana Tran, Westminster, CA (US)

(21) Appl. No.: 19/451,559

(22) Filed: Jan. 16, 2026

Related U.S. Application Data
(63) Continuation of application No. PCT/US2024/037016, filed on Jul. 8, 2024.
(60) Provisional application No. 63/514,333, filed on Jul. 18, 2023.

(51) Int. Cl. A61F 2/24

(52) U.S. Cl. CPC A61F 2/2418; A61F 2/2436; A61F 2250/0069; A61F 2250/0098

(57) ABSTRACT

An implant may include an expandable frame configured to expand from a compressed state to an expanded state when deployed, where the expandable frame includes one or more vertical struts that each include a span that remains in a longitudinal orientation in both the compressed state and the expanded state, and where at least one of the vertical struts includes a first set of lateral openings extending partway through the at least one vertical strut.

### CROSS REFERENCE TO RELATED APPLICATIONS

The present application is a continuation of International Application No. PCT/US 2024/037016, filed Jul. 8, 2024, which claims the benefit of U.S. Provisional Ser. No. 63/514,333 , filed Jul. 18, 2023, both of which applications are incorporated by reference herein in their entirety.

### FIELD

The present disclosure relates to implantable adaptor systems for engaging and retaining a prosthetic implant such as a prosthetic heart valve in a lumen of the body, such as a blood vessel or valve of the heart.

### BACKGROUND

Prosthetic heart valves may be used to treat cardiac valvular disorders. The native heart valves (the aortic, pulmonary, tricuspid, and mitral valves) function to prevent backward flow or regurgitation, while allowing forward flow. These heart valves may be rendered less effective by congenital, inflammatory, infectious conditions, etc. Such conditions may eventually lead to serious cardiovascular compromise or death. For many years, doctors attempted to treat such disorders with surgical repair or replacement of the valve during open heart surgery.

A transcatheter technique for introducing and implanting a prosthetic heart valve using a catheter in a manner that is less invasive than open heart surgery may reduce complications associated with open heart surgery. In this technique, a prosthetic valve may be mounted in a crimped state on the end portion of a catheter and advanced through a blood vessel of the patient until the valve reaches the implantation site. The valve at the catheter tip may then be expanded to its functional size at the site of the defective native valve, such as by inflating a balloon on which the valve is mounted or, for example, the valve may have a resilient, self-expanding stent or frame that expands the valve to its functional size when it is advanced from a delivery sheath at the distal end of the catheter. Optionally, the valve may have a balloon-expandable frame, self-expanding frame, a mechanically-expandable frame, and/or a frame expandable in multiple or a combination of ways.

Transcatheter heart valves (THVs) may be appropriately sized for placement inside many native cardiac valves or orifices. However, with larger native valves, blood vessels (e.g., an enlarged aorta), grafts, etc., aortic transcatheter valves might be too small to secure into the larger implantation or deployment site. In this case, the transcatheter valve may not be large enough to sufficiently expand inside the native valve or other implantation or deployment site or the implantation/deployment site may not provide a good seat for the THV to be secured in place. As one example, aortic insufficiency may be associated with difficulty securely implanting a THV in the aorta and/or aortic valve. Accordingly, there exists a need for improved systems and methods of securing a THV in a relatively large diameter blood vessel or annulus.

### SUMMARY

Certain embodiments of the disclosure pertain to docking stations, frame adaptors, prestents, and the like for engaging and retaining a prosthetic implant such as a prosthetic heart valve in a lumen of the body, such as a blood vessel or valve of the heart. In a representative embodiment, an implant may include an expandable frame configured to expand from a compressed state to an expanded state when deployed, where the expandable frame includes one or more vertical struts that each include a span that remains in a longitudinal orientation in both the compressed state and the expanded state, and where at least one of the vertical struts includes a first set of lateral openings extending partway through the at least one vertical strut.

The foregoing and other objects, features, and advantages of the described technology will become more apparent from the following detailed description, which proceeds with reference to the accompanying figures.

### BRIEF DESCRIPTION OF THE DRAWINGS

FIG. 1A is a cutaway view of the human heart in a diastolic phase.

FIG. 1B is a cutaway view of the human heart in a systolic phase.

FIG. 2A is a cutaway view of the human heart with an embodiment of an example docking station positioned in a blood vessel, the inferior vena cava (IVC).

FIG. 2B is an end view of an example docking station and valve showing the valve in an open configuration such that blood may flow through the valve, e.g., when the heart is in a diastolic phase.

FIG. 2C is an end view of the docking station and valve of FIG. 2B showing the valve in a closed configuration, e.g., when the heart is in a systolic phase.

FIG. 3A is a sectional view of an example embodiment of a docking station with an example transcatheter valve disposed inside the docking station.

FIG. 3B is a top view of the docking station and valve illustrated by FIG. 3A.

FIG. 3C is a perspective view of an example embodiment of a docking station that illustrates an example of a frame portion that may be used in the docking station of FIGS. 3A-3B.

FIG. 3D is a sectional view of the docking station illustrated by FIG. 3A where the transcatheter valve shown is representative of a leaflet type transcatheter valve.

FIGS. 4A and 4B schematically illustrate deployment of a docking station.

FIGS. 4C and 4D schematically illustrate deployment of a valve in the docking station.

FIG. 5 illustrates components of another example implant configured to dock and/or support one or more prosthetic valves and/or valve components.

FIG. 6 illustrates components of an additional example implant configured to dock and/or support one or more prosthetic valves and/or valve components.

FIG. 7A illustrates a side view of components of another example implant configured to dock and/or support one or more prosthetic valves and/or valve components.

FIGS. 7B-7F illustrate an example deployment of the implant illustrated in FIG. 7A.

FIG. 8 illustrates a view of an example implant configured to dock and/or support one or more prosthetic valves and/or valve components when cut or etched from a cylindrical tube.

FIG. 9A illustrates a front view of a portion of the implant of FIG. 8 when in a compressed state.

FIG. 9B illustrates a close up of a front view of a portion of the implant of FIG. 8 when in the compressed state.

FIG. 9C illustrates a top-down view of a portion of the implant of FIG. 8 when in an expanded state.

FIG. 9D illustrates a front view of another portion of the implant of FIG. 8 when in the compressed state.

FIG. 9E illustrates a front view of a portion of the implant of FIG. 8 when in the expanded state.

FIG. 10 illustrates a portion of another example implant configured to dock and/or support one or more prosthetic valves and/or valve components when in a compressed state.

FIGS. 11A-11C illustrate an example of flexion of a frame of an implant in side-to-side flexion.

FIGS. 11D-11F illustrate an example of flexion of the frame of the implant of FIGS. 11A-11C in front-to-back flexion.

FIGS. 12A and 12B illustrate additional examples of lateral openings of an implant.

FIG. 13 illustrates a view of another example implant configured to dock and/or support one or more prosthetic valves and/or valve components when cut or etched from a cylindrical tube.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

It should be understood that the disclosed embodiments may be adapted for delivery and implantation in any of the native annuluses and blood vessels of the heart (e.g., the pulmonary, mitral, and tricuspid annuluses, the inferior and superior vena cava, etc.), and may be used with any of various delivery approaches (e.g., retrograde, antegrade, transseptal, transventricular, transatrial, etc.).

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, may be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

In the context of the present application, the terms “lower” and “upper” are used interchangeably with the terms “inflow” and “outflow”, respectively. Thus, for example, typically the lower end of a valve or docking station as depicted in the figures is its inflow end and the upper end of the valve or docking station is its outflow end unless explicitly described otherwise.

As used herein, the term “proximal” refers to a position, direction, or portion of a device that is closer to the user (e.g., clinician) and further away from the implantation site and/or body lumen orifice. As used herein, the term “distal” refers to a position, direction, or portion of a device that is further away from the user and closer to the implantation site and/or body lumen orifice. Thus, for example, proximal motion of a device is motion of the device toward the user, while distal motion of the device is motion of the device away from the user.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part may become a “lower” part simply by turning the object over. Nevertheless, it is still the same part and the object remains the same. As used herein, “and/or” means “and” or “or”, as well as “and” and “or”.

As used herein, the terms “integrally formed” and “unitary construction” refer to a construction that does not require any sutures, fasteners, or other securing means to attach two portions of the construction together.

Examples of Disclosed Technology

The present disclosure pertains to valve adapter/docking station/landing zone/prestent technology for implanting a prosthetic heart valve, such as a transcatheter heart valve, in a lumen or valve of the heart where the diameter of the lumen or valve is significantly greater than the functional diameter of the prosthetic valve. In certain examples, the docking station may comprise a radially expandable and collapsible frame formed from a plurality of struts, and including a valve seat within the frame configured to receive an expandable prosthetic valve. In certain embodiments, the valve seat may comprise a plurality of struts coupled to the frame and angled inwardly toward the longitudinal axis of the frame. The valve seat may be configured to engage and retain prosthetic valves of a variety of types and sizes. The outer aspect of the docking station frame may engage the surrounding tissue of the native lumen and form a seal, and the valve seat may engage and retain the prosthetic heart valve within the docking station. In certain embodiments, the frame may comprise a sealing member configured to form a seal between the frame and the surrounding anatomy without substantially interfering with blood flow entering the upstream portions of the frame, such as adjacent the ostia of the hepatic veins when implanted in the inferior vena cava.

In certain embodiments, the struts of the valve seat may form valve seat frame cells of the frame. In certain embodiments, struts and/or cells of the valve seat may comprise free end portions/apices, which may be disposed within the lumen of the docking station frame and define a reduced diameter portion configured to engage and retain a prosthetic heart valve. In certain embodiments, the struts of the valve seat may be coupled to the docking station frame at frame junctions, and the free end portions/apices of the valve seat may be offset from the frame junctions in a downstream direction toward the outflow end of the frame. This reduce or minimize the length of the prosthetic valve that protrudes or extends distally or in the downstream direction from the docking station. In certain embodiments, the struts of the valve seat may be wholly disposed within the docking station frame, or the free end portions/apices of the valve seat may define a downstream-most end of the docking station frame.

In certain embodiments, the docking station frame may comprise a plurality of circumferentially-arranged vertical struts. The vertical struts may reduce or prevent foreshortening of the frame between the collapsed and expanded configuration. This may facilitate more accurate and/or predictable deployment of the docking station from the collapsed delivery configuration. The vertical struts may also facilitate recapture of the docking station frame from a partially deployed state by limiting an angle formed by the flared, partially deployed portion of the frame and the longitudinal axis of the delivery apparatus. The vertical struts may also strengthen the frame and reduce or eliminate infolding or invagination of the frame during recapture.

In certain embodiments, the docking station frame may comprise a plurality of free end portions or apices arranged circumferentially around the frame. In certain embodiments, the free apices may be located between pairs of adjacent vertical struts. In certain embodiments, the free apices may be proximal and/or distal apices of frame cells defined between pairs of longitudinal frame struts. In certain embodiments, the frame cells may be axially spaced apart from each other. The free apices may be configured to engage the surrounding tissue of a body lumen in which the docking station frame is implanted to prevent frame movement/migration/rotation relative to the body lumen.

In some embodiments, docking stations/devices for prosthetic valves or THVs are illustrated as being used within the superior vena cava (SVC), inferior vena cava (IVC), or both the SVC and the IVC, although the docking stations/devices (e.g., docking station/device 10, other docking stations/devices described herein, modified versions of the docking stations, etc.) may be used in other areas of the anatomy, heart, or vasculature, such as the tricuspid valve, the pulmonary valve, the pulmonary artery, the aortic valve, the aorta, the mitral valve, or other locations. The docking stations/devices described herein may be configured to compensate for the deployed transcatheter valve or THV being smaller and/or having a different geometrical shape than the space (e.g., anatomy/heart/vasculature/etc.) in which it is to be placed. For example, the native anatomy (e.g., the IVC) may be oval, egg shaped, or another shape, while the prosthetic valve or THV may be cylindrical.

Various embodiments of docking stations/devices and examples of prosthetic valves or transcatheter valves are disclosed herein, and any combination of these options may be made unless specifically excluded. For example, any of the docking stations/devices disclosed, may be used with any type of valve, and/or any delivery system, even if a specific combination is not explicitly described. Likewise, the different constructions and features of docking stations/devices and valves may be mixed and matched, such as by combining any docking station type/feature, valve type/feature, covering/sealing element, etc., even if not explicitly disclosed. In short, individual components of the disclosed systems may be combined unless mutually exclusive or physically impossible.

For the sake of uniformity, in the present disclosure the docking stations are typically depicted such that the right atrium end (e.g., the outflow end) is up, while the ventricular end or IVC end (e.g., the inflow end) is down unless otherwise indicated.

### First Representative Embodiment

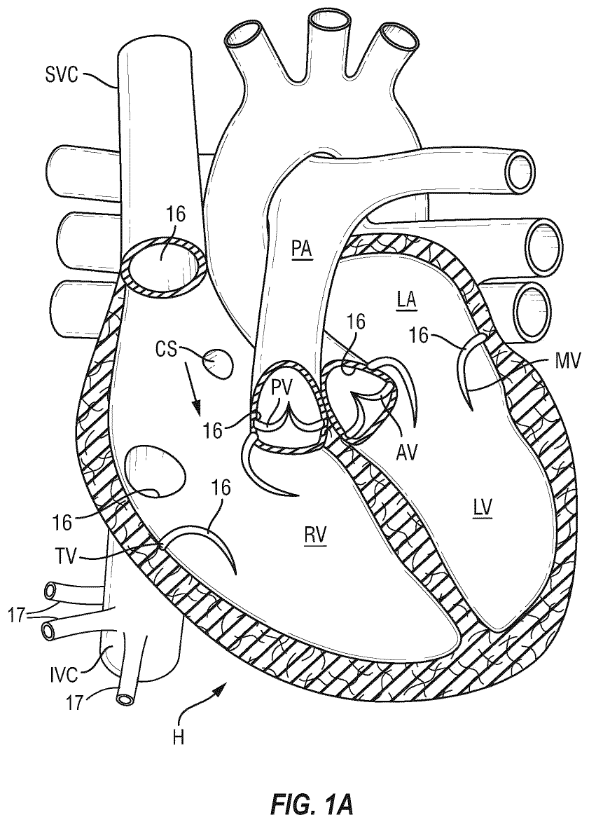

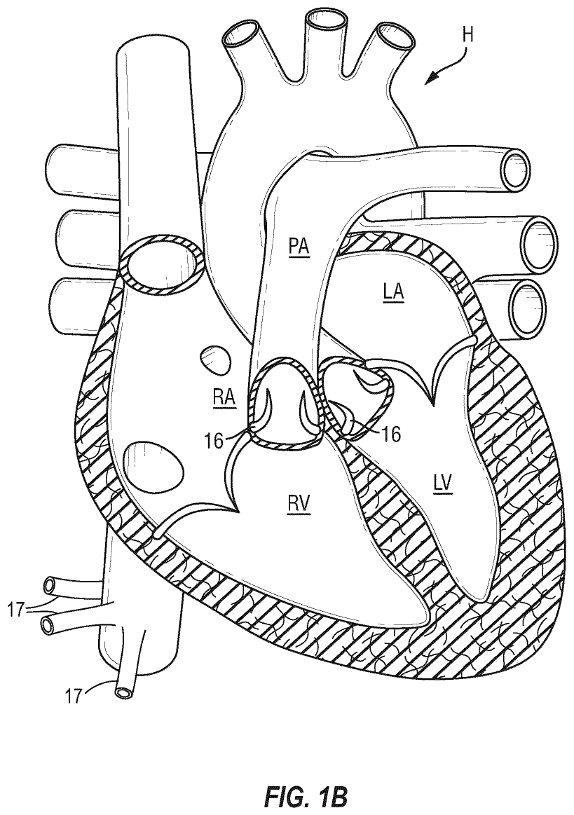

FIGS. 1A and 1B are cutaway views of the human heart (H) in diastolic and systolic phases, respectively. The right ventricle (RV) and left ventricle (LV) are separated from the right atrium (RA) and left atrium (LA), respectively, by the tricuspid valve (TV) and the mitral valve (MV); i.e., the atrioventricular valves. Additionally, the aortic valve (AV) separates the left ventricle (LV) from the ascending aorta (not identified) and the pulmonary valve (PV) separates the right ventricle from the pulmonary artery (PA). Each of these valves has flexible leaflets extending inward across the respective orifices that come together or “coapt” in the flowstream to form one-way, fluid-occluding surfaces. The docking stations and valves of the present application are described, for illustration, primarily with respect to the inferior vena cava (IVC), superior vena cava (SVC), and aorta/aortic valve. A defective aortic valve, for example, may be a stenotic aortic valve and/or suffer from insufficiency and/or regurgitation. The blood vessels, such as the aorta, IVC, SVC, pulmonary artery, may be healthy or may be dilated, distorted, enlarged, have an aneurysm, or be otherwise impaired. Anatomical structures of the right atrium RA, right ventricle RV, left atrium LA, and left ventricle LV will be explained in greater detail. The devices described herein may be used in various areas whether explicitly described herein or not, e.g., in the IVC and/or SVC, in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The hepatic veins 17 carry blood from the liver to the inferior vena cava IVC. The coronary sinus (CS) is a collection of veins joined together to form a large vessel that collects deoxygenated blood from the heart muscle (myocardium), and delivers it to the right atrium RA. During the diastolic phase, or diastole, seen in FIG. 1A, the deoxygenated blood from the IVC, SVC, and CS that has collected in the right atrium RA passes through the tricuspid valve TV and into the RV as the right ventricle RV expands. In the systolic phase, or systole, seen in FIG. 1B, the right ventricle RV contracts to force the deoxygenated blood collected in the RV through the pulmonary valve PV and pulmonary artery into the lungs.

The devices described herein may be used to supplement the function of a defective tricuspid valve and/or to prevent too much pressure from building up in the RA. During systole, the leaflets of a normally functioning tricuspid valve TV close to prevent the venous blood from regurgitating back into the right atrium RA. When the tricuspid valve does not operate normally, blood may backflow or regurgitate into the right atrium RA, the inferior vena cava IVC, the superior vena cava SVC, and/or other vessels in the systolic phase. Blood regurgitating backward into the right atrium increases the volume of blood in the atrium and the blood vessels that direct blood to the heart. This may cause the right atrium to enlarge and cause blood pressure to increase in the right atrium and blood vessels, which may cause damage to and/or swelling of the liver, kidneys, legs, other organs, etc. A transcatheter valve (THV) implanted in the inferior vena cave IVC and/or the superior vena cava SVC may prevent or inhibit blood from backflowing into the inferior vena cave IVC and/or the superior vena cava SVC during the systolic phase.

The length L, diameter D, and curvature or contour may vary greatly between the superior vena cava SVC and inferior vena cava IVC of different patients. The relative orientation and location of the IVC and/or SVC may also vary between patients. Further, the size or diameter D may vary significantly along the length L of an individual IVC and/or SVC. Also, the anatomy of the IVC and/or SVC is soft, flexible, and dynamic as compared to other cardiac vessels, such as the aorta. This softer, more flexible, and/or more dynamic (moving and/or shape changing) characteristic of the IVC and SVC make it more difficult for a transcatheter valve frame or a docking station that supports a transcatheter valve to anchor in the IVC and/or the SVC than in the aorta. Further, other regions or other vasculature in other areas of the body and across patients where docking stations could be used may also vary significantly in shape and size.

The left atrium LA receives oxygenated blood from the left and right pulmonary veins, which then travels through the mitral valve to the left ventricle. During the diastolic phase, or diastole, seen in FIG. 1A, the oxygen rich blood that collects in the left atrium LA passes through the mitral valve MV and into the left ventricle LV as the left ventricle LV expands. In the systolic phase, or systole, seen in FIG. 1B, the left ventricle LV contracts to force the oxygen rich blood through the aortic valve AV and aorta into the body through the circulatory system. In certain embodiments, the devices described herein may be used to supplement or replace the function of a defective aortic valve. For example, the devices herein may be particularly effective for treating aortic insufficiency. During diastole, the leaflets of a normally functioning aortic valve AV close to prevent the oxygen rich blood from regurgitating back into the left ventricle LV. When the aortic valve does not operate normally, blood backflows or regurgitates into the left ventricle LV. A THV implanted in the aortic valve helps prevent or inhibit blood from backflowing into the left ventricle LV during the diastole phase. The length L, diameter, D, and curvature or contour of the aortic root may vary greatly between different patients, especially if the aorta is dilated, distorted, or enlarged. Further, the size or diameter D may vary significantly along the length L of an individual aorta.

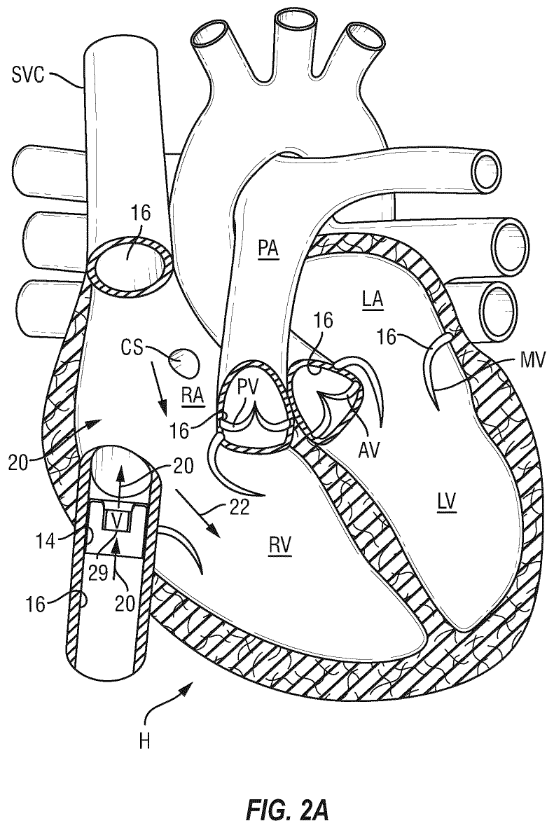

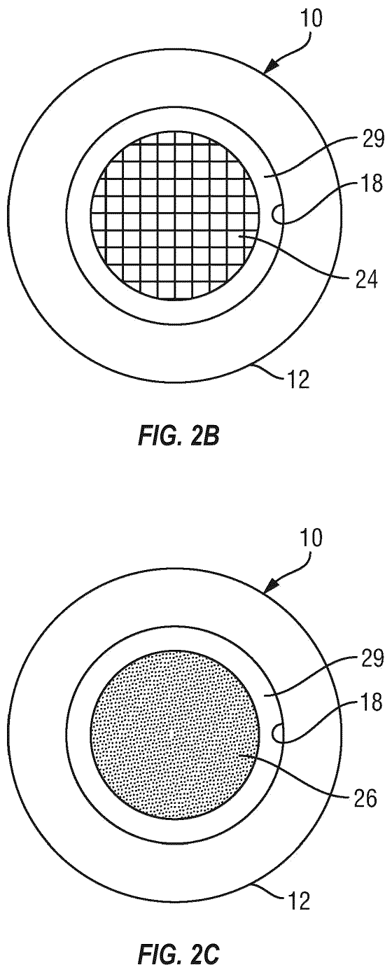

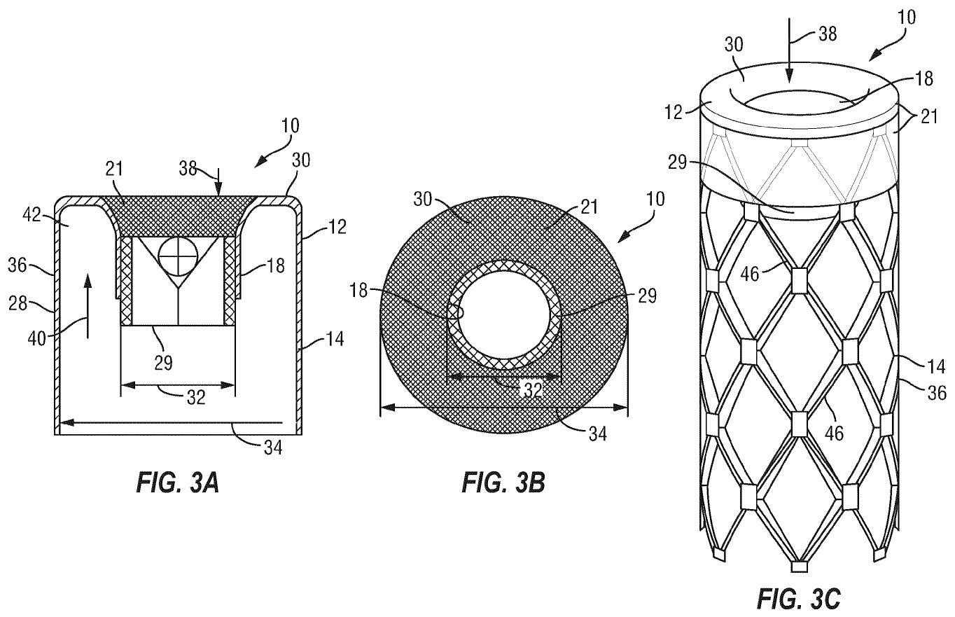

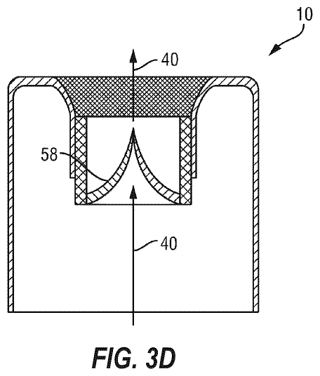

Referring to FIGS. 2A, 3A, 3B, and 3C, in some embodiments an expandable docking station/device/valve adapter/landing zone/prestent 10 includes one or more sealing portions 12, a valve seat 18, and one or more retaining portions 14. The sealing portion(s) 12 provide a seal between the docking station 10 and an interior surface 16 (see FIG. 2A) of the circulatory system. The valve seat 18 provides a supporting surface for implanting or deploying a valve 29 in the docking station 10 after the docking station 10 is implanted in the circulatory system. Optionally, the docking station 10 and the valve 29 may be integrally formed, for example, in one embodiment, the valve seat 18 may be omitted. When integrally formed, the docking station 10 and the valve 29 may be deployed as a single device, rather than first deploying the docking station 10 and then deploying the valve 29 into the docking station. Any of the docking stations and/or valve seats 18 described herein may be provided or formed with an integrated valve 29.

The retaining portion 14 helps retain the docking station 10 and the valve 29 at the implantation position or deployment site in the circulatory system. The retaining portion 14 may take a wide variety of different forms. In some embodiments, the retaining portion 14 includes friction enhancing features that reduce or eliminate migration of the docking station 10. The friction enhancing features may take a wide variety of different forms. For example, the friction enhancing features may comprise barbs, spikes, texturing, adhesive, and/or a cloth or polymer cover with high friction properties on the retaining portions 14. Such friction enhancing features may also be used on any of the various docking stations or retaining portions described herein.

Expandable docking station 10 and valve 29 as described in the various embodiments herein are also representative of a variety of docking stations and/or valves described herein or that might be known or developed, e.g., a variety of different types of valves could be substituted for and/or used as valve 29 in the various docking stations.

FIGS. 2A, 2B, and 2C illustrate a representative example of the operation of the docking stations 10 and valves 29 disclosed herein. In the example of FIGS. 2A, 2B, and 2C, the docking station 10 and valve 29 are deployed in the inferior vena cava IVC. However, the docking station 10 and valve 29 may be deployed in any interior surface within the heart or a lumen of the body. For example, the various docking stations and valves described herein may be deployed in the superior vena cava SVC, the tricuspid valve TV, the pulmonary valve PV, pulmonary artery, the mitral valve MV, the aortic valve AV, aorta, or other vasculature/lumens in the body.

FIGS. 2A and 2B illustrate the valve 29, docking station 10 and heart H, when implanted in the IVC and the heart H is in the diastolic phase. When the heart is in the diastolic phase, the valve 29 opens. Blood flows from the inferior vena cava IVC and the superior vena cava SVC, into the right atrium RA. The blood that flows from the inferior vena cava IVC flows through the docking station 10 and valve 29 as indicated by arrows 20. Also, while in the diastolic phase, blood in the right atrium flows through the tricuspid valve TV, and into the right ventricle RV and valve as indicated by arrows 22. FIG. 2B illustrates space 24 that represents the valve 29 being open when the heart is in the diastolic phase. A variety of types of valves may be used that may open and close in a variety of ways (e.g., including valves with leaflets of tissue that open then coapt to close), so the drawings are meant to be representative of a variety of valves that may operate in different ways. FIG. 2B does not show the interface between the docking station 10 and the inferior vena cava to simplify the drawing. The cross-hatching in FIG. 2B represents blood flow through the valve 29. In some embodiments, blood is prevented or inhibited from flowing between the inferior vena cava IVC and the docking station 10 by the sealing portion 12 and blood is prevented or inhibited from flowing between the docking station 10 and the valve by implanting or seating the valve in the seat 18 of the docking station 10. In this example, blood only substantially flows or is only able to flow through the valve 29 when the valve is open (e.g., in certain embodiments, only when the heart is in the diastolic phase).

FIG. 2C illustrates the valve 29 and docking station 10, when the valve 29 is closed (e.g., when implanted in the IVC and the heart H is in the systolic phase). When implanted in the IVC and the heart is in the systolic phase, the valve 29 closes. Blood is prevented from flowing from the right atrium RA into the inferior vena cava IVC by the valve 29 being closed. As such, the closed valve 29 prevents any blood that regurgitates through the tricuspid valve TV during the systolic phase from being forced into the inferior vena cava IVC. The solid area 26 in FIG. 2C represents the valve 29 being closed (e.g., in certain embodiments, when the heart is in the systolic phase). FIG. 2C is meant to be representative of a variety of valves, even though those valves may close in different ways.

In some embodiments, the docking station 10 acts as an isolator that prevents or substantially prevents radial outward forces of the valve 29 from being transferred to the inner surface 16 of the circulatory system. In one embodiment, the docking station 10 includes a valve seat 18 that resists expansion, e.g., is not expanded radially outwardly (e.g., the diameter of the valve seat does not increase) or is not substantially expanded radially outward (e.g., the diameter of the valve seat increases by less than 4 mm) by the radially outward force of the transcatheter valve or valve 29. The valve seat may be configured such that expansion of a THV/valve 29 increases the diameter of the valve seat only to a diameter less than an outer diameter of the docking station 10 when the docking station is implanted. Retaining portions 14 and sealing portions 12 may be configured to impart only relatively small radially outward forces on the inner surface 16 of the circulatory system (as compared to the radially outward force applied to the valve seat 18 by the valve 29). Having a valve seat 18 that is stiffer or less radially expansive than the outer portions of the docking station (e.g., retaining portions 14 and sealing portions 12), as in the various docking stations described herein, provides many benefits, including allowing a THV/valve 29 to be implanted in vasculature or tissue of varying strengths, sizes, and/or shapes. The outer portions of the docking station may better conform to the anatomy (e.g., vasculature, tissue, heart, etc.) without putting too much pressure on the anatomy, while the THV/valve 29 may be firmly and securely implanted in the valve seat 18 with forces that will prevent or mitigate the risk of migration or slipping.

The docking station 10 may include any combination of one or more than one different types of valve seats 18, retaining portions 14, and/or sealing portions 12. For example, the valve seat 18 may be a separate component that is attached to the frame 28 of the docking station 10, while the sealing portion is integrally formed with the frame 28 of the docking station. Also, the valve seat 18 may be a separate component that is attached to the frame 28 of the docking station 10, while the sealing portion 12 is a separate component that is also attached to the frame 28 of the docking station. Optionally, the valve seat 18 may be integrally formed with the frame 28 of the docking station 10, while the sealing portion is integrally formed with the frame 28 of the docking station. Further, the valve seat 18 may be integrally formed with the frame 28 of the docking station 10, while the sealing portion is a separate component that is attached to the frame 28 of the docking station 10.

The sealing portion 12, the valve seat 18, and one or more retaining portions 14 of the various docking stations herein may take a variety of different forms and characteristics. In FIGS. 3A-3C, an expandable frame 28 provides the shape of the sealing portion 12, the valve seat 18, and the retaining portion 14. The expandable frame 28 may take a wide variety of different forms. The illustrated expandable frame 28 in FIGS. 3A-3C has an end 30 having an inside diameter 32 and an outside diameter 34. An annular or cylindrical outer portion or wall 36 extends downward from the outside diameter 34 of the end 30. An annular or cylindrical valve seat 18 or wall extends downward from the inside diameter 32 of the end 30. In the illustrated example, the expandable frame 28 is an expandable lattice. The expandable lattice may be made in a variety of ways, e.g., with individual wires connected to form the lattice, braiding, cut from a sheet and then rolled or otherwise formed into the shape of the expandable frame, molded, cut from a cylindrical tube (e.g., cut from a nitinol tube), other ways, or a combination of these.

The frame 28 may be made from a highly flexible metal, metal alloy, or polymer. Examples of metals and metal alloys that may be used include, but are not limited to, nitinol and other shape memory alloys, elgiloy, and stainless steel, but other metals and highly resilient or compliant non-metal materials may be used to make the frame 28. These materials may allow the frame to be compressed to a small size, and then when the compression force is released, the frame will self-expand back to its pre-compressed diameter and/or the frame may be expanded by inflation of a device positioned inside the frame. The frame 28 may also be made of other materials and be expandable and collapsible in different ways, e.g., mechanically-expandable, balloon-expandable, self-expandable, or a combination of these.

The sealing portions may take a wide variety of different forms. In the example of FIGS. 3A-3C, a covering/material 21 is attached to a portion of the frame 28 to form the sealing portion 12. However, the sealing portion 12 may be formed in a wide variety of other ways. The covering/material 21 may be a fabric material, polymer material, or other material. The sealing portion 12 may take any form that prevents or inhibits the flow of blood from flowing around the outside surface of the valve 29 and through the docking station. In the example of FIGS. 3A, 3B, and 3C, the sealing portion 12 comprises a covering/material 21 (e.g., a fabric or other covering material that may be the same as or similar to other coverings/materials described herein) that extends up to the valve seat 18. The covering/material 21 may be shaped and positioned in a variety of ways, e.g., the covering/material may be configured to partially cover the valve seat 18, entirely cover the valve seat 18, or not cover the valve seat 18 when the frame 28 is expanded. The covering/material 21 (e.g., fabric or other covering material) that forms the sealing portion 12 may also extend radially outward, covering the end 30 of the frame 28, and may optionally extend (e.g., longitudinally, downward, etc.) to cover at least a portion of the annular outer portion or wall 36. The sealing portion 12 provides a seal between the docking station 10 and an interior surface 16 (see FIG. 2) of the circulatory system. That is, the sealing portion 12 and the closed valve 29 prevent or inhibit blood from flowing in the direction indicated by arrow 38. In the example of FIGS. 3A and 3B, blood is not inhibited from flowing in the direction indicated by arrow 40 into the area 42 between the valve seat 18 and the annular outer portion or wall 36.

The valve seat may take a wide variety of different forms. The valve seat 18 is a portion of the frame 28 in the example of FIGS. 3A-3C. However, the valve seat 18 may be formed separately from the frame 28. The valve seat 18 may take any form that provides a supporting surface for implanting or deploying a valve 29 in the docking station 10 after the docking station 10 is implanted in the circulatory system. The valve seat may optionally be reinforced with a reinforcing material (e.g., a suture, wire, band, collar, etc. that may circumscribe the valve seat or a portion of the valve seat). The valve 29 is schematically illustrated in FIG. 3A to indicate that the valve 29 may take a wide variety of different forms. FIG. 3D illustrates the more specific example where the valve 29 is a leaflet type THV, such as the SAPIEN®3 valve available from Edwards Lifesciences Corporation, including a plurality of leaflets 58. In some embodiments, a valve 29 is integrated with or replaces the valve seat 18, such that docking station 10 is configured as a transcatheter valve that is delivered as a single unit in the same step (as opposed to first implanting a docking stations and subsequently implanting a separate valve/THV in the docking station). Optionally, any of the docking stations described herein may be formed as a valve or THV, e.g., with valve tissue or other valve material integrated into the docking station.

The retaining portions 14 may take a wide variety of different forms. For example, the retaining portion(s) 14 may be any structure that sets the position of the docking station 10 in the circulatory system. For example, the retaining portion(s) 14 may press against or into the inside surface 16 or contour/extend around anatomical structures of the circulatory system to set and maintain the position of the docking station 10. The retaining portion(s) 14 may be part of or define a portion of the body and/or sealing portion of the docking station 10 or the retaining portion(s) 14 may be a separate component that is attached to the body of the docking station. The docking station 10 may include a single retaining portion 14 or two, or more than two retaining portions. The retaining portion(s) 14 may include friction enhancing features as discussed above.

In the example of FIGS. 3A-3C, the retaining portion 14 comprises the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the circulatory system to retain the docking station 10 and the valve 29 at the implantation position. In the illustrated embodiment, the retaining portion 14 is elongated to allow a relatively small force to be applied to a large area of the interior surface 16, while the valve 29 may apply a relatively large force to the valve seat 18. For example, the length of the retaining portion 14 may be twice, three times, four times, five times, or greater than five times the outside diameter of the transcatheter valve. Applying a small radially outward force over a larger area may be sufficient to securely hold the docking station in place, and this design/configuration may allow the docking station to conform to the unique shape/size of the anatomy and avoid/reduce the likelihood of damaging relatively weaker native tissue. Thereby the valve 29 may be securely held in a variety of locations and anatomies (e.g., the docking station of FIGS. 3A-D is usable in the IVC, SVC, aorta, etc.).

In certain examples, the retaining portion 14 may comprise the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the aorta to retain the docking station 10 and the valve 29 at the implantation position. In certain examples, the shape set may also be selected to substantially match the shape of a portion of the aorta. The retaining portion 14 may be elongated to allow a relatively small force to be applied to a large area of the interior surface 16, while the valve 29 may apply a relatively large force to the valve seat 18, as discussed above.

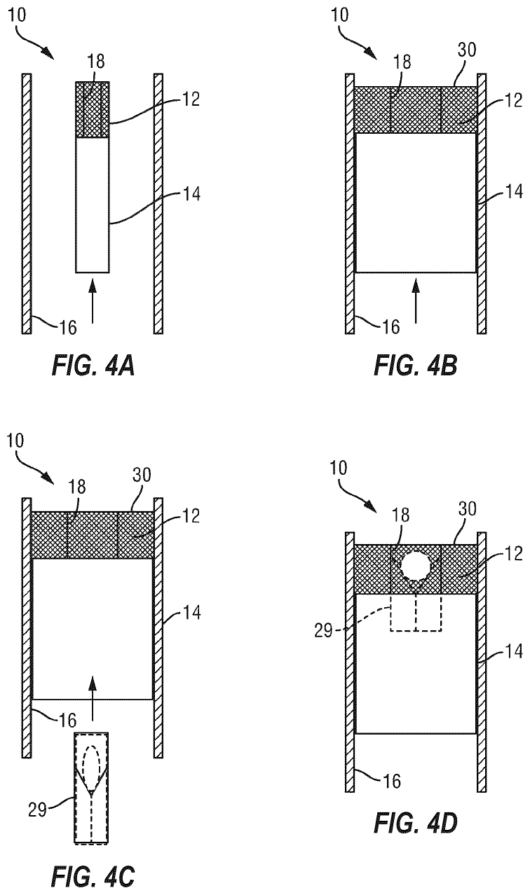

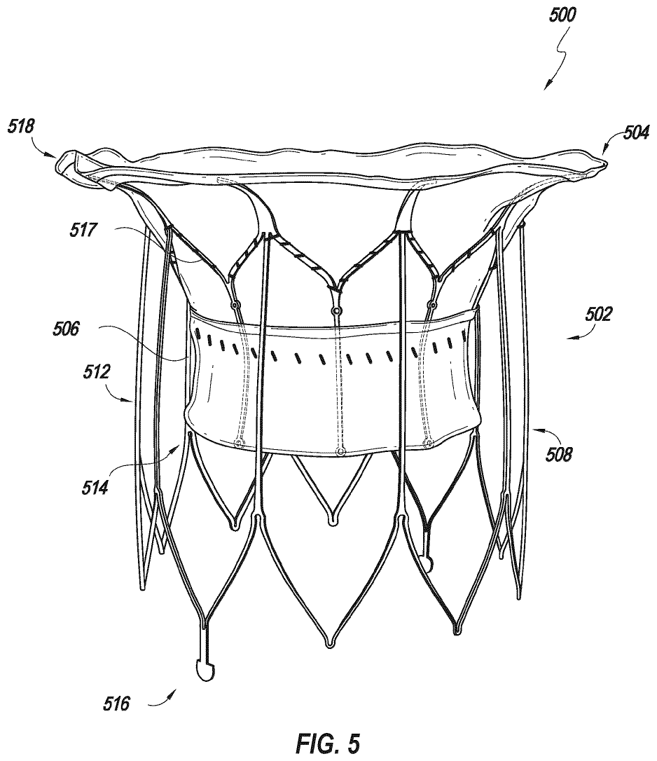

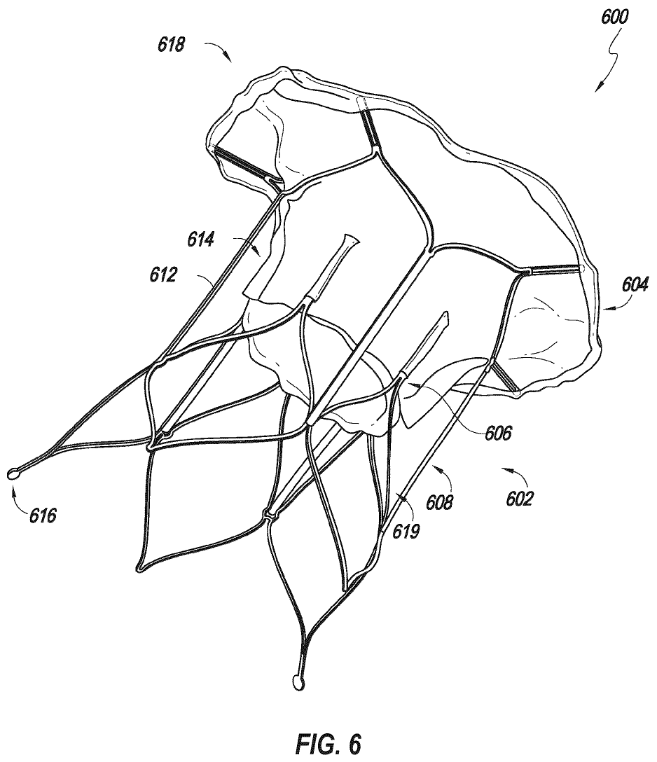

FIGS. 4A-4D schematically illustrate an example deployment of the docking station 10 and valve 29 in the circulatory system. Referring to FIG. 4A, the docking station 10 is in a compressed form/configuration and is introduced to a deployment site in the circulatory system. For example, the docking station 10, may be positioned at a deployment site in a SVC, IVC, aorta, or other location. Referring to FIG. 4B, the docking station 10 is expanded in the circulatory system such that the sealing portion(s) 12 and the retaining portion(s) 14 engage the inside surface 16 of a portion of the circulatory system. The docking station may be self-expanding, and may be advanced from a delivery capsule into the expanded state, or plastically expandable such that it may be expanded using a balloon or other expansion device. Referring to FIG. 4C, after the docking station 10 is deployed, the valve 29 is in a compressed form and is introduced into the valve seat 18 of the docking station 10. Referring to FIG. 4D, the valve 29 is expanded in the docking station, such that the valve 29 engages the valve seat 18 and the seat 18 of the docking station supports the valve. The docking station 10 allows the valve 29 to operate within the expansion diameter range for which it is designed. In the examples depicted herein, the docking station 10 is longer than the valve. However, in some embodiments the docking station 10 may be the same length or shorter than the length of the valve 29. Similarly, the valve seat 18 may be longer, shorter, or the same length as the length of the valve 29. Any of the docking station embodiments described herein may be deployed in the manner described above.

### Second Representative Embodiment

FIG. 5 illustrates components of another example implant 500 configured to dock and/or support one or more prosthetic valves and/or valve components in accordance with one or more instances. The implant 500 may comprise a frame 502 and/or a sealing element 504, which may include a skirt, covering, and/or similar device. The frame 502 may be configured to form an inner frame 506 and/or an outer frame 508. The inner frame 506 may form a first diameter that is less than a second diameter of the outer frame 508. The inner frame 506 and outer frame 508 may be extensions of a common device and/or may extend from each other.

The frame 502 may comprise a wire frame formed by a network of struts 512 (e.g., wires, cords, and/or bars) forming one or more cells 514. The frame 502 may mostly comprise empty space and/or cells 514 between the struts 512. For example, the struts 512 may be generally thin and/or may be spaced apart to create relatively large gaps between the struts 512, as shown in FIG. 5. In some examples, the outer frame 508 may comprise a series of longitudinally extending struts 512 aligned in series and/or in parallel around a circumference of the implant 500. Each of the struts 512 may join to one or more adjacent struts 512 at a proximal end 516 of the implant 500 and/or may join with one or more adjacent struts 512 and/or with the inner frame 506 at a distal end 518 of the implant 500.

The inner frame 506 may similarly comprise a network of generally thin, elongate, and/or spaced apart struts 512. In some examples, one or more struts 512 of the inner frame 506 may extend generally in series and/or in parallel longitudinally along a length of the implant 500. The one or more struts 512 of the inner frame 506 may be disposed between struts 512 of the outer frame 508.

In some examples, the outer frame 508 may have a generally cylindrical form and/or may at least partially enclose a complete circumference of the inner frame 506 and/or of a portion of the sealing element 504. The outer frame 508 may comprise a network of struts 512, which may include wires, arms, bars, cords, walls, and/or similar components forming one or more cells 514 and/or openings through the outer frame 508. The one or more cells 514 may be configured to allow blood flow through the outer frame 508. The cells 514 may have any suitable shape and/or size. The outer frame 508 may form generally elongate cells 514 extending approximately an entire length of the outer frame 508 and/or from a proximal end 516 to a distal end 518 of the implant 500. The one or more cells 514 may have triangular forms at end points of the one or more cells 514. However, the one or more struts 512 may form cells 514 having different shapes. For example, the struts 512 may be configured to form generally rectangular and/or diamond-shaped cells 514. While the struts 512 are shown having generally thick structures, the one or more struts 512 may have wire-like and/or generally thin forms. In some examples, the inner frame 506 and/or outer frame 508 may be configured to maintain a uniform structure and/or strut 512 pattern along a length of the frame 502.

The implant 500 may be configured for delivery and/or placement at an SVC and/or IVC of a heart. For example, the implant 500 may be configured for placement at or near an in-flow junction of the SVC and/or IVC to the right atrium of the heart.

At a distal end 518 of the implant 500, the outer frame 508 and inner frame 506 may join together and/or the inner frame 506 may extend away from the outer frame 508 and/or along an inner lumen formed by the outer frame 508. In some examples, the outer frame 508 and inner frame 506 may both have a generally flared and/or conical form at or near the distal end 518 of the implant 500. The inner frame 506 may have a flaring angle that is greater than the outer frame 508 such that the diameter of the inner frame 506 may be less than the diameter of the outer frame 508. The inner frame 506 may be configured to extend along at least a portion of the length of the outer frame 508.

The flared end (e.g., distal end 518) of the outer frame 508 and/or inner frame 506 may be configured to engage an atrium and/or other chamber when implanted. The sealing element 504 may be configured to extend along the inner frame 506 and/or may be configured to wrap around the outer frame 508 and/or extend between the outer frame 508 and the native tissue. The outer frame 508 may be configured to extend into the IVC and/or other blood vessel and/or the sealing element 504 may be configured to extend from the outer frame 508 to an inner surface of the inner frame 506. The sealing element 504 may be configured for engagement with a prosthetic valve and/or other implant. For example, the sealing element 504 may be configured to provide a mounting surface for a prosthetic valve and/or may be configured to securely hold the prosthetic valve. The sealing element 504 may be configured to extend along only a portion of the implant 500 such that the sealing element 504 may not extend across one or more branching vessels of the blood vessel.

In some examples, the implant 500 may comprise one or more outward bulbs configured to extend outwardly from the diameter of the outer frame 508 to facilitate anchoring of the implant 500 within a blood vessel.

The frame 502 may comprise one or more downward-extending arms 517 (e.g., extending towards the proximal end 516) configured to form the inner frame 506. For example, the arms 517 may extend downwardly from the outer frame 508 at or near the distal end 518 of the frame 502. The one or more arms 517 may be configured to extend at an acute angle away from the outer frame into the lumen of the frame 502 and/or may extend generally in parallel with the outer frame 508 at or near distal ends of the one or more arms 517.

FIG. 6 illustrates components of another example implant 600 configured to dock and/or support one or more prosthetic valves and/or valve components in accordance with one or more instances. The implant 600 may comprise a frame 602 and/or a sealing element 604, which may include a skirt, covering, and/or similar device. The frame 602 may be configured to form an inner frame 606 and/or an outer frame 608. The inner frame 606 may form a first diameter that is less than a second diameter of the outer frame 608. The inner frame 606 and outer frame 608 may be extensions of a common device and/or may extend from each other.

The frame 602 may comprise a wire frame formed by a network of struts 612 (e.g., wires, cords, and/or bars) forming one or more cells 614. Such struts 612 may be referred to as vertical struts. The frame 602 may mostly comprise empty space and/or cells 614 between the struts 612. For example, the struts 612 may be generally thin and/or may be spaced apart to create relatively large gaps between the struts 612, as shown in FIG. 6. In some examples, the outer frame 608 may comprise a series of longitudinally extending struts 612 aligned in series and/or in parallel around a circumference of the implant 600. Each of the struts 612 may join to one or more adjacent struts 612 at a proximal end 616 of the implant 600 and/or may join with one or more adjacent struts 612 and/or with the inner frame 606 at a distal end 618 of the implant 600.

The inner frame 606 may similarly comprise a network of generally thin, elongate, and/or spaced apart struts 612. In some examples, one or more struts 612 of the inner frame 606 may extend generally in series and/or in parallel longitudinally along a length of the implant 600. The one or more struts 612 of the inner frame 606 may be disposed between struts 612 of the outer frame 608.

In some examples, the outer frame 608 may have a generally cylindrical form and/or may at least partially enclose a complete circumference of the inner frame 606 and/or of a portion of the sealing element 604. The outer frame 608 may comprise a network of struts 612, which may include wires, arms, bars, cords, walls, and/or similar components forming one or more cells 614 and/or openings through the outer frame 608. The one or more cells 614 may be configured to allow blood flow through the outer frame 608. The cells 614 may have any suitable shape and/or size. The outer frame 608 may form generally elongate cells 614 extending approximately an entire length of the outer frame 608 and/or from a proximal end 616 to a distal end 618 of the implant 600. The one or more cells 614 may have triangular forms at end points of the one or more cells 614. However, the one or more struts 612 may form cells 614 having different shapes. For example, the struts 612 may be configured to form generally rectangular and/or diamond-shaped cells 614. While the struts 612 are shown having generally thick structures, the one or more struts 612 may have wire-like and/or generally thin forms. In some examples, the inner frame 606 and/or outer frame 608 may be configured to maintain a uniform structure and/or strut 612 pattern along a length of the frame 602.

The implant 600 may be configured for delivery and/or placement at an SVC and/or IVC of a heart. For example, the implant 600 may be configured for placement at or near an in-flow junction of the SVC and/or IVC to the right atrium of the heart.

At a distal end 618 of the implant 600, the outer frame 608 and inner frame 606 may join together and/or the inner frame 606 may extend away from the outer frame 608 and/or along an inner lumen formed by the outer frame 608. In some examples, the outer frame 608 and inner frame 606 may both have a generally flared and/or conical form at or near the distal end 618 of the implant 600. The inner frame 606 may have a flaring angle that is greater than the outer frame 608 such that the diameter of the inner frame 606 may be less than the diameter of the outer frame 608. The inner frame 606 may be configured to extend along at least a portion of the length of the outer frame 608.

The flared end (e.g., distal end 618) of the outer frame 608 and/or inner frame 606 may be configured to engage an atrium and/or other chamber when implanted. The sealing element 604 may be configured to extend along the inner frame 606 and/or may be configured to wrap around the outer frame 608 and/or extend between the outer frame 608 and the native tissue. The outer frame 608 may be configured to extend into the IVC and/or other blood vessel and/or the sealing element 604 may be configured to extend from the outer frame 608 to an inner surface of the inner frame 606. The sealing element 604 may be configured for engagement with a prosthetic valve and/or other implant. For example, the sealing element 604 may be configured to provide a mounting surface for a prosthetic valve and/or may be configured to securely hold the prosthetic valve. The sealing element 604 may be configured to extend along only a portion of the implant 600 such that the sealing element 604 may not extend across one or more branching vessels of the blood vessel.

In some examples, the implant 600 may comprise one or more outward bulbs configured to extend outwardly from the diameter of the outer frame 608 to facilitate anchoring of the implant 600 within a blood vessel.

The frame 602 may comprise one or more upward-extending arms 619 (e.g., extending towards the distal end 618) configured to form the inner frame 606. For example, the arms 619 may extend downwardly from the outer frame 608 at or near the distal end 618 of the frame 602. The one or more arms 619 may be configured to extend at an acute angle away from the outer frame into the lumen of the frame 602 and/or may extend generally in parallel with the outer frame 608 at or near distal ends of the one or more arms 619.

The arms 619 of the implant 600 may extend upwardly from the proximal end 616 to the distal end 618. For example, the one or more arms 619 may extend at an approximately 45-degree angle from the outer frame 608 at or near the distal end 618 of the implant 600 and/or may extend upwardly generally in parallel with the outer frame 608 along a midsection of the implant 600.

### Third Representative Embodiment

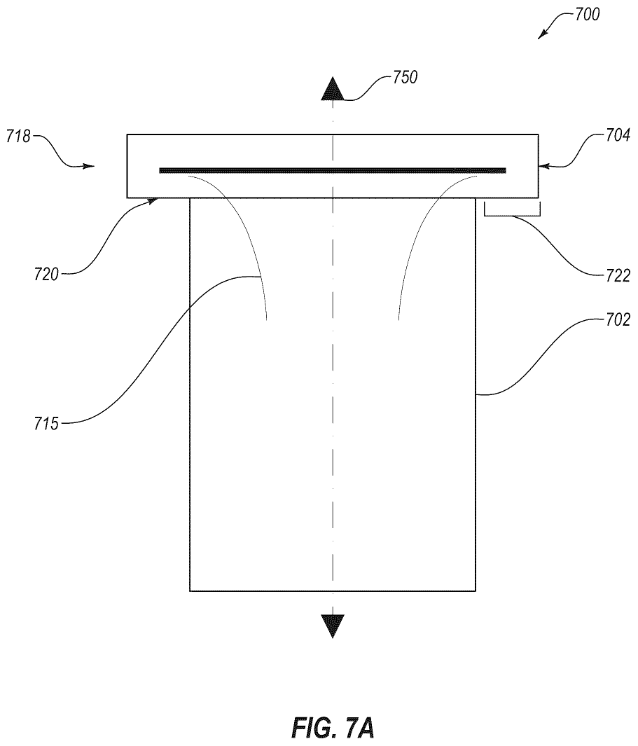

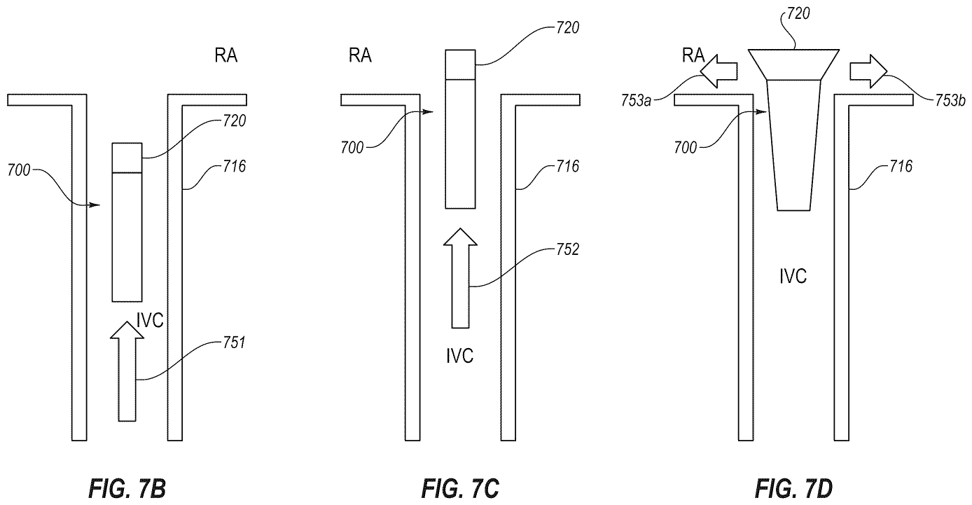

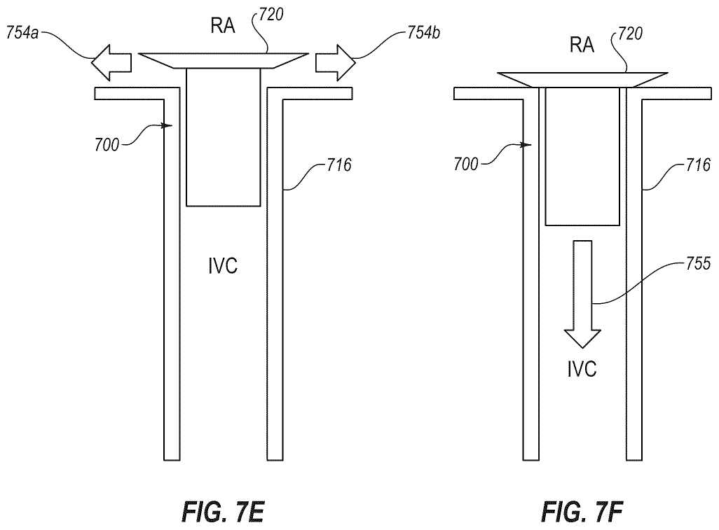

FIG. 7A illustrates a side view of components of another example implant 700 configured to dock and/or support one or more prosthetic valves and/or valve components in accordance with one or more embodiments of the present disclosure. The implant 700 may include a frame 702 and/or a sealing element 704, which may include a skirt, covering, and/or similar device. The frame 702 may be configured to form a valve seat 715. The valve seat 715 may be disposed within a central cavity created by the frame 702. At a distal end 718 of the implant 700, the implant may include a flange 720 to facilitate the sealing element 704 sealing against the anatomy of a patient. For convenience, a central axis 750 of the implant 700 is also illustrated in FIG. 7A.

The flange 720 may provide a projection that extends beyond the remainder of the frame 702. For example, as illustrated in FIG. 7, the flange 720 may produce a lip 722 that is shaped or configured to abut anatomy of the patient. For example, rather than being disposed wholly within the IVC (such as the embodiment illustrated in FIG. 2A), the distal end 718 of the implant 700 may extend into the right atrium with the flange 720 resting against the sidewall of the right atrium proximate where the IVC enters the right atrium (RA). By providing the lip 722, a better seal may be created by the sealing element 704 as compared to embodiments that are radially pressed against the IVC, such as that illustrated in FIG. 2A.

In some embodiments, the shape or profile of the frame 702 or other components forming the flange 720 or flared end at the distal end 718 of the implant 700 may include a similar or comparable profile to that illustrated in FIGS. 5 and/or 6. For example, the frame 702 may include one or more cells with arms extending beyond the remainder of the frame 702 at the distal end 718 of the implant 700.

In some embodiments, the flange 720 may be generally normal to the central axis 750. Additionally or alternatively, the flange 720 may be generally centered around the line depicting the central axis 750.

FIGS. 7B-7F illustrate an example deployment of the implant 700 illustrated in FIG. 7A. The implant 700 may traverse a circulatory system 716 of a patient to arrive at a target deployment location.

As illustrated in FIG. 7B, a catheter (not illustrated) carrying the implant 700 may traverse the circulatory system 716 towards a deployment location. For example, as illustrated in FIG. 7B, it may be desired to deploy the implant 700 between the IVC and the RA. The implant 700 may traverse the circulatory system 716 in a distal direction conveyed by the arrow 751. In some embodiments, the implant 700 may be included in a delivery capsule or may be deployable via a balloon or other deployment mechanism.

As illustrated in FIG. 7C, the implant 700 may begin to pass through the IVC and into the RA as it continues along the distal direction of travel conveyed by the arrow 752.

As illustrated in FIG. 7D, upon reaching the deployment or target location, the implant 700 may be either partially or fully deployed. For example, a sheath or outer covering of the delivery capsule associated with the catheter may be moved proximally relative to the implant 700, exposing the flange 720 and/or other portions of the implant 700. The radial expansion of the implant 700 during deployment is conveyed by the arrows 753a and 753b. While illustrated as being partially within the RA and partially within the IVC, it will be appreciated that the implant 700 may be fully within the RA when it is partially or fully deployed.

As illustrated in FIG. 7E, the implant 700 may be deployed until the flange 720 is substantially or fully deployed. The continued deployment is illustrated by the arrows 754a and 754b. In some embodiments, the implant 700 may be deployed until a radial edge of the flange 720 extends beyond the circumference of the IVC. Such deployment may include full or partial deployment of the flange 720 and/or full or partial deployment of the implant 700.

In some embodiments, the stage of deployment illustrated in FIG. 7E may include the implant 700 being at least 30% deployed, at least 40% deployed, at least 50% deployed, at least 60% deployed, at least 70% deployed, at least 80% deployed, at least 90% deployed, or 100% deployed (or fully deployed). Additionally or alternatively, the stage of deployment illustrated in FIG. 7E may include the flange 720 portion of the implant 700 being at least 30% deployed, at least 40% deployed, at least 50% deployed, at least 60% deployed, at least 70% deployed, at least 80% deployed, at least 90% deployed, or 100% deployed (or fully deployed).

As illustrated in FIG. 7F, after the stage of deployment illustrated in FIG. 7E is reached, the catheter may be drawn back in the proximal direction as illustrated by the arrow 755. Doing so may facilitate the flange 720 seating against the wall of the RA to create a seal.

In some embodiments, after seating the flange 720 against the wall of the RA, the implant 700 may be deployed the remainder of the way to be fully deployed. Additionally, the implant 700 may be disengaged from the catheter and the catheter may be retrieved from the body of the patient. For example, a coupling foot of the frame 702 may be decoupled from an attachment feature of the delivery capsule. Additionally or alternatively, after the implant 700 is fully deployed, the valve may traverse the circulatory system 716 of the patient and be affixed to the implant 700 (such as by being coupled to a catheter in a valve delivery capsule, traversing the circulatory system 716 of the patient until the valve may be deployed from the valve delivery capsule and affixed to the implant 700, and the catheter and valve delivery capsule retrieved).

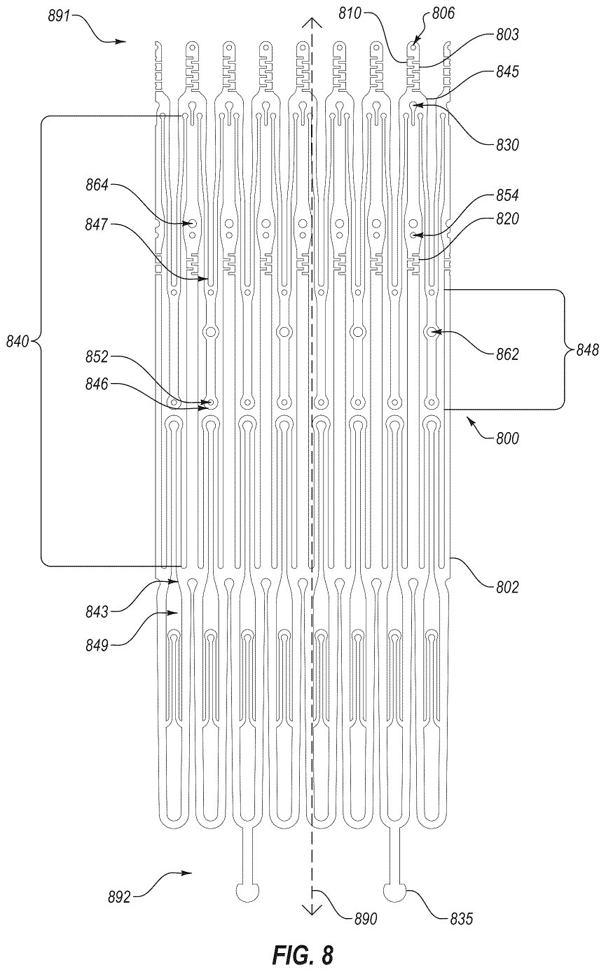

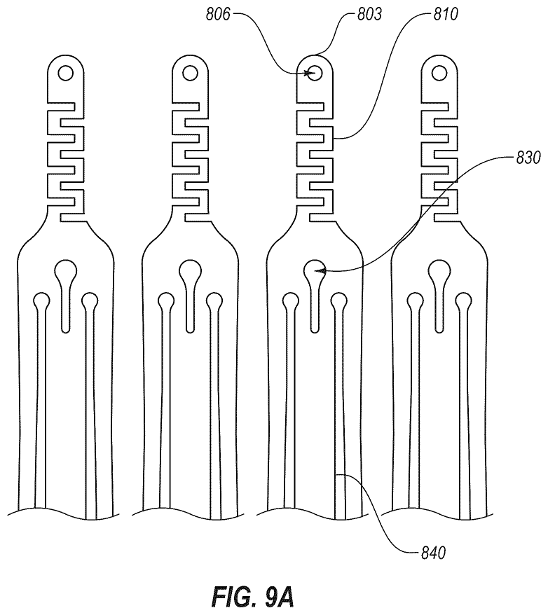

FIG. 8 illustrates a view of an example implant 800 configured to dock and/or support one or more prosthetic valves and/or valve components. The implant 800 includes an expandable frame 802 that is configured to expand from a compressed state into an expanded state when deployed. The view of FIG. 8 is in the compressed stated. The view illustrated in FIG. 8 depicts the implant 800 when cut or etched from a cylindrical tube, and for convenience in visualization, the tube is sliced in a longitudinal direction and unrolled such that an outer surface of the implant 800 that had a tubular shape is in a single plane. It will be appreciated that in implementation, the tube may be cut or etched and remain in the tubular shape. As illustrated in FIG. 8, the implant 800 may be cut or etched in a compressed state to facilitate the implant 800 traversing the body of the patient to the delivery location (e.g., the IVC, right atrium, etc.).

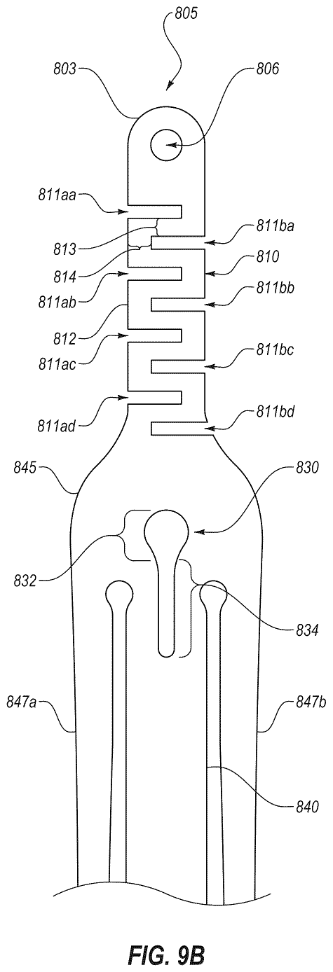

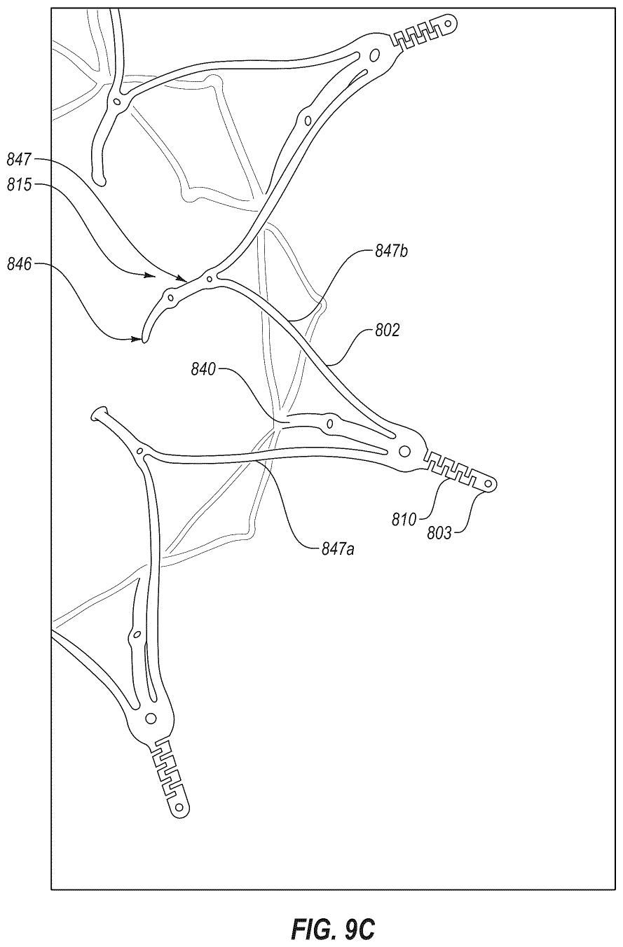

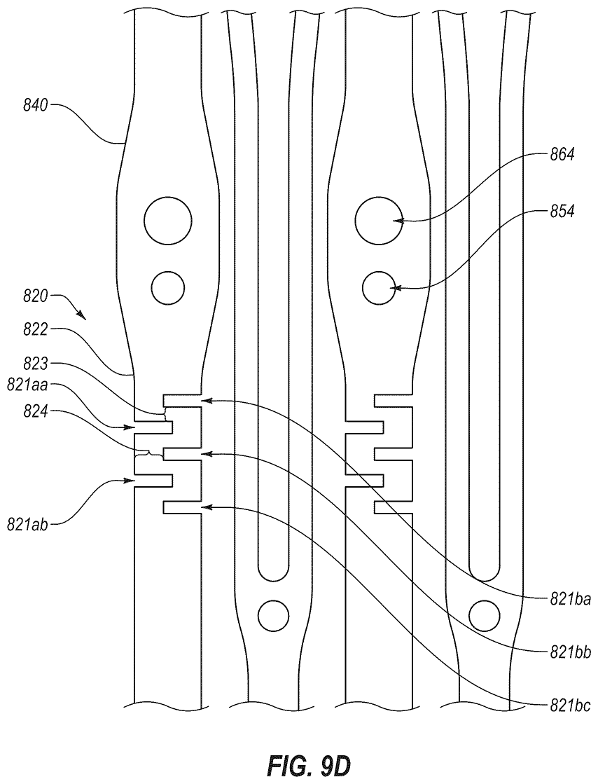

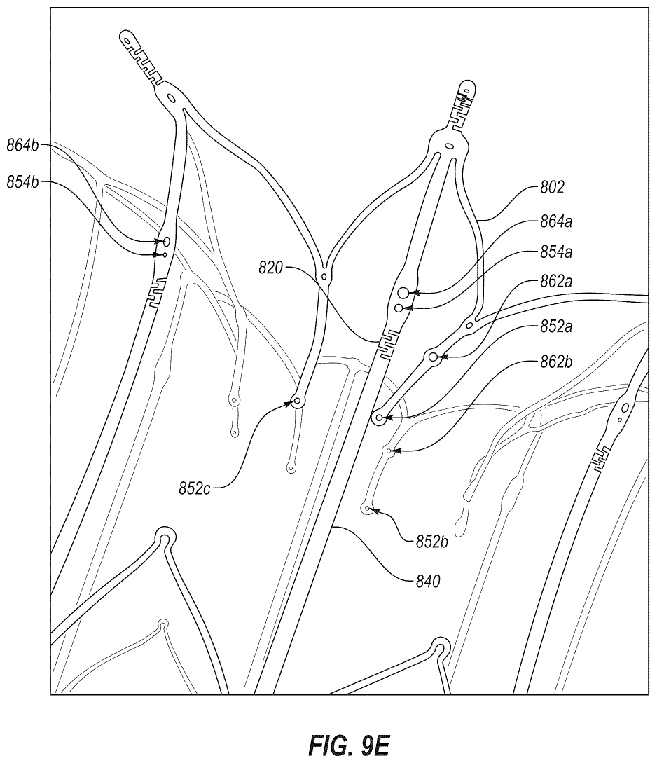

FIG. 9A illustrates a front view of a portion of the implant of FIG. 8 when in a compressed state. FIG. 9B illustrates a close up of a front view of a portion of the implant of FIG. 8 when in the compressed state. FIG. 9C illustrates a top-down view of a portion of the implant of FIG. 8 when in an expanded state. FIG. 9D illustrates a front view of another portion of the implant of FIG. 8 when in the compressed state. FIG. 9E illustrates a front view of a portion of the implant of FIG. 8 when in the expanded state.

The implant 800 may be similar or comparable in some regards to one or more of the implants 500 of FIGS. 5 and/or 600 of FIG. 6. For example, the implant 800 may include a valve seat 815, which may be comparable or similar to the valve seat 18 and/or 715. As illustrated in FIGS. 8 and 9A-9E, various features may be added to a frame 802 of the implant 800 to improve the flexibility of the implant 800. For example, one or more flange struts 803 and/or vertical struts 840 may include a first set of lateral openings 810 or a second set of lateral openings 820, respectively, to facilitate flexion or other movement of the respective struts. As an additional example, the frame 802 may include stress/strain-relieving cutouts or other openings, such as a keyhole opening 830 to facilitate deformation of the frame 802 without material failure, such as when going from the compressed state to the expanded state.

The flange struts 803 may facilitate the formation of a flange of the implant 800 when in the expanded state. Examples of such flanges are illustrated in FIGS. 5, 6, and 7A-7E. For example, when the implant 800 changes to an expanded state (such as that illustrated in FIGS. 9C and/or 9E), the flange struts 803 may project in a generally radial direction away from a central, longitudinal axis 890 of the implant 800. In these and other embodiments, the flange struts 803 may extend away from the frame 802 at or near an end of a vertical strut 840 of the implant.

In some embodiments, the flange struts 803 may project away from the longitudinal axis 890 in a plane normal to the longitudinal axis 890. Additionally or alternatively, the flange struts 803 may extend away from the frame 802 at an angle to such a plane. Additionally or alternatively, the flange struts 803 may curve away from such a plane, and may or may not end with a relatively linear portion that is parallel to such a plane or at an angle to such a plane.

The flange struts 803 may have a sealing element (not shown) that extends over the flange struts 803 to create a seal against the anatomy of a patient and against a valve ultimately deployed within the implant 800. The sealing element may be comparable or similar to sealing element 504, 604, and/or 704 illustrated in FIGS. 5, 6, and/or 7A-7E, respectively.

In some embodiments, a distal end 805 of the flange struts 803 may include an eyelet 806 or other opening to facilitate attachment of the sealing element into the implant 800. For example, a skirt or cloth may be sutured or sewn onto the implant 800 via the eyelets 806 of the flange struts 803. In some embodiments, each of the flange struts 803 may include eyelets 806. Additionally or alternatively, less than all of the flange struts 803 may include eyelets 806, such as every other flange strut 803, every third flange strut 803, among others.

In some embodiments, other eyelets may be included to facilitate attachment of the sealing element into the implant 800. For example, the implant 800 may include internal eyelets 852 (such as the internal eyelets 852a-c) proximate a terminal point 846 of arms 847 extending from shoulders 845 of the frame 802. For example, two adjacent arms 847 may converge through a portion 848 and terminate at the terminal point 846. The internal eyelets 852 may facilitate suturing or sewing the sealing element to the implant 800 within the valve seat 815.

In some embodiments, the implant 800 may include external eyelets 854 (such as the external eyelets 854a and 854b) disposed on the vertical struts 840. The external eyelets 854 may be located proximate other openings, such as those within which radiopaque material may be coined. In some embodiments, the external eyelets 854 may be disposed on the vertical struts between the shoulder 845 and the second set of lateral openings 820. In some embodiments, the external eyelets 854 may be located closer to the second set of lateral openings 820 than the shoulder 845.

With reference to FIG. 9B, the first set of lateral openings 810 may alternate which edge of the flange strut 803 from which the lateral openings originate and may extend through a majority or more of a width of the flange strut 803. For example, as illustrated in FIG. 9B, a first lateral opening 811aa may originate from the left, a second lateral opening 811ba may originate from the right, a third lateral opening 811ab may originate from the left, a fourth lateral opening 811bb may originate from the right, a fifth lateral opening 811ac may originate from the left, a sixth lateral opening 811bc may originate from the right, a seventh lateral opening 811ad may originate from the left, and an eighth lateral opening 811bd may originate from the right. In these and other embodiments, the material 812 of the flange strut 803 within which the first set of lateral openings 810 are formed may result in a zig-zag shape in the material 812. While illustrated as a squared zig-zag, it will be appreciated that a rounded zig-zag, a chevron zig-zag, or any other form of alternating pattern is contemplated by the present disclosure.

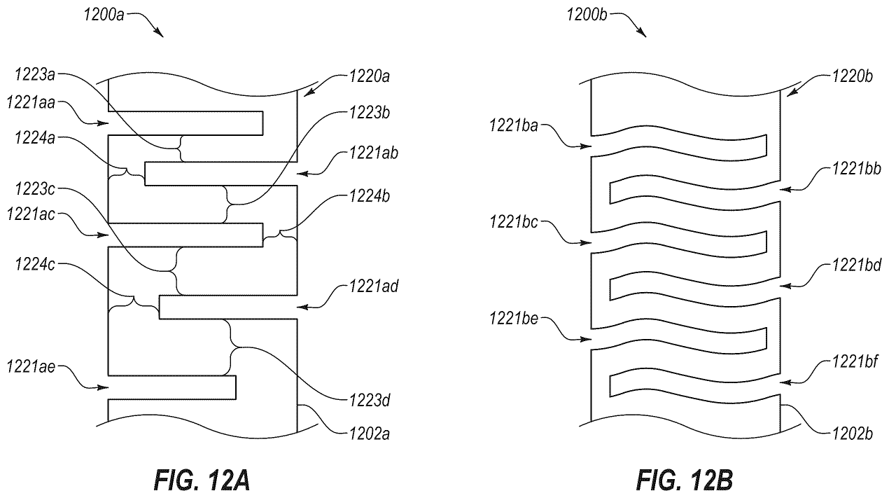

In some embodiments, a lateral portion 813 of the material may be narrower than a longitudinal portion 814 of the material. In some embodiments, each lateral portion 813 may be the same size as the other lateral portions 813 of the material. Additionally or alternatively, the lateral portions 813 may be of varying sizes and/or shapes, examples of which will be described with reference to FIGS. 12A and/or 12B. In some embodiments, variations in the lateral portions 813 and/or the longitudinal portions 814 may determine how far across a width of the flange strut 803 the first set of lateral openings 810 may extend. In some embodiments, the longitudinal portions 814 may be sized such that the first set of lateral openings 810 extend for at least 40% of the width of the flange strut 803, at least 50% of the width of the flange strut 803, at least 60% of the width of the flange strut 803, at least 70% of the width of the flange strut 803, at least 80% of the width of the flange strut 803, and/or at least 90% of the width of the flange strut 803.

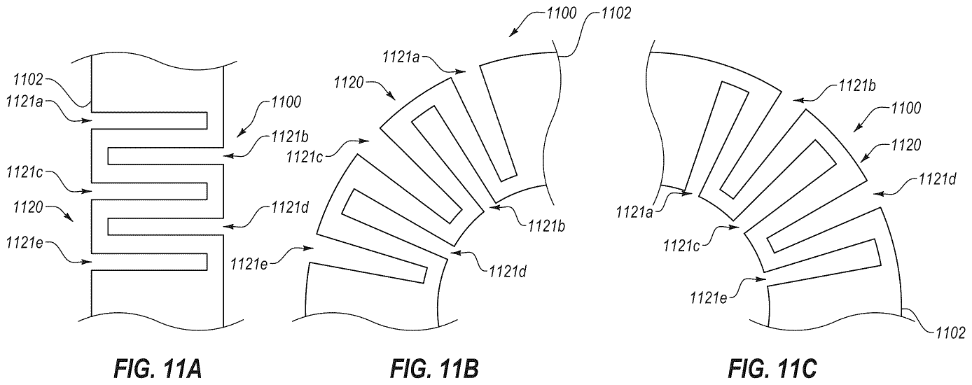

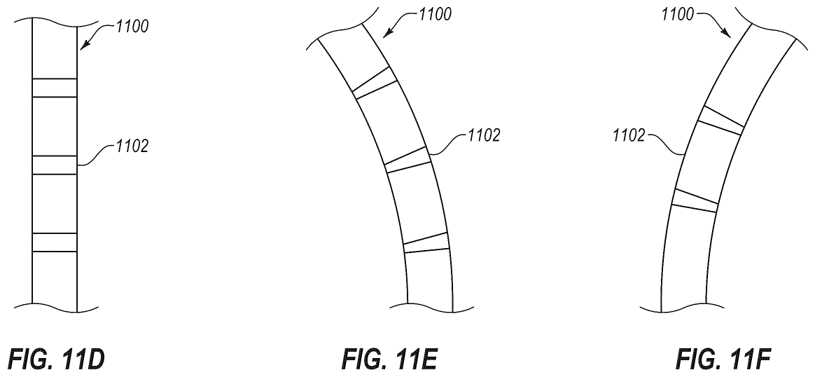

In some embodiments, the shape of the first set of lateral openings 810 may permit flexion of the flange strut 803 in at least four directions in a plane normal to the longitudinal axis 890 when in the compressed state. For example, the flange strut 803 may flex to either side as the lateral openings are closed on one edge of the flange strut 803 and expanded on the opposite side. Continuing the example, for the left side, the lateral openings 811aa, 811ab, 811ac, and 811ad may be closed at the left edge and the lateral openings 811ba, 811bb, 811bc, and 811bd may be opened more widely at the right edge as the flange strut 803 flexes to the left. Further continuing the example, for the right side, the lateral openings 811ba, 811bb, 811bc, and 811bd may be closed at the right edge and the lateral openings 811aa, 811ab, 811ac, and 811ad may be opened more widely at the left edge as the flange strut 803 flexes to the right. Additionally or alternatively, the flange strut 803 may flex to the front or the back (e.g., into or out of the page as illustrated in FIGS. 8, 9A, and 9B) by slight deformations in the material 812 as the zig-zag shape flexes to the front or the back. Examples of the side-to-side flexion is illustrated in FIGS. 11A-11C and examples of the front to back flexion is illustrated in FIGS. 11D-11F.

In some embodiments, the first set of lateral openings 810 may include an equal number of openings originating on one side as originate on the other side. For example, the first set of lateral openings 810 may include four openings originating at the left and four openings originating at the right. In some embodiments, the first set of lateral openings 810 may include an unequal number of openings originating at the sides, for example, one side may include one more opening than the other side, or more. For example, the first set of lateral openings 810 may include three openings on one side and two on the other, although any number and combination of lateral openings is contemplated. In some embodiments, the number of first set of lateral openings 810 may be selected based on a tradeoff between material integrity and rigidity compared to the increased flexibility and deformability of the flange struts 803 as more lateral openings are included.

In some embodiments, the first set of lateral openings 810 may span from a shoulder 845 of the frame 802 towards a distal end of the flange strut 803. In some embodiments, the first set of lateral openings 810 may cover a majority of the flange strut 803. In some embodiments, the first set of lateral openings 810 may cover at least 20%, 30%, 40%, 50%, 60%, 70%, 80%, 90%, or more of the span of the flange strut 803 from the shoulder 845 to the distal end of the flange strut 803.

In some embodiments, all of the flange struts 803 include the first set of lateral openings 810. In some embodiments, fewer than all of the flange struts 803 include the first set of lateral openings 810. For example, every other flange strut 803, every third flange strut 803, every fourth flange strut 803, etc. may not include the first set of lateral openings 810. Additionally or alternatively, every other flange strut 803, every third flange strut 803, every fourth flange strut 803, etc. may include the first set of lateral openings 810.

By providing greater flexibility in the flange struts 803 of the implant 800, the implant 800 may be better able to create a seal against the anatomy of the patient. For example, the flexibility granted from the flange struts 803 may permit the flange to conform more readily to anatomy and/or for a clinician to use an increased force in the longitudinal direction (e.g., such as illustrated in FIG. 7F) to better create a seal without damaging the tissue of the patient.

In some embodiments, the implant 800 may include one or more radiopaque markers (not illustrated) to facilitate guidance, orientation, and otherwise facilitate a procedure of guiding the implant 800 through the body of the patient to the target location, deploying the implant 800, and/or otherwise positioning the implant 800 in the desired position. The radiopaque markers may facilitate a view into an axial or radial orientation, state of deployment, or other information for the clinician when performing a procedure involving the implant 800. For example, a first set of openings 862 (such as the openings 862a and 862b) within which a first set of radiopaque markers may be disposed may be located in the arm 847 to facilitate visualization of the location of the valve seat 815. As another example, a second set of openings 864 (such as the openings 864a and 864b) within which a second set of radiopaque markers may be disposed may be located in the vertical struts 840 to facilitate visualization of the location of the outer seal. In these and other embodiments, radiopaque material may be coined into the first set of openings 862 and/or the second set of openings 864, such as tantalum, bismuth, iodine, barium, or gold.

When performing a procedure, the clinician may utilize the first set of radiopaque markers associated with the openings 862 to visualize the location of the valve seat 815. For example, using fluoroscopy or other visualization techniques, the clinician may verify that the valve seat 815 is in the target location relative to the anatomy of the patient. Additionally or alternatively, the clinician may utilize the second set of radiopaque markers associated with the openings 864 to visualize the location of the flange and/or the outer seal between the implant 800 and the anatomy of the patient. For example, using fluoroscopy or other visualization techniques, the clinician may verify that a distal portion of the vertical struts 840 are properly oriented in the target location relative to the anatomy of the patient.

In some embodiments, the first set of openings 862 may be located along the arms 847 towards a terminal point 846 in the arms 847. For example, as illustrated in FIGS. 9C and 9E, the arms 847 may flex inwards to facilitate formation of the valve seat 815. In these and other embodiments, the first set of openings 862 may be located between the shoulder 845 and the terminal point 846 of the arms 847. In some embodiments, the first set of openings 862 may be closer to the terminal point 846 than the shoulder 845. In some embodiments, the first set of openings 862 may be disposed in a portion 848 of the arm 847 within which two adjacent arms have joined or merged. In these and other embodiments, the first set of openings 862 may be disposed in a flare of material in the arm 847 to facilitate the first set of openings 862. In some embodiments, the first set of openings 862 may be located at a consistent longitudinal position relative to the longitudinal axis 890.

In some embodiments, the second set of openings 864 may be disposed in the vertical struts 840 between the second set of lateral openings 820 and the shoulder 845. In some embodiments, the openings 864 may be located closer to the lateral openings 820 than they are to the shoulder 845. In some embodiments, the second set of openings 864 may be located at a consistent longitudinal position relative to the longitudinal axis 890.

In some embodiments, each of the portions 848 and/or the vertical struts 840 may include the radiopaque markers. Additionally or alternatively, fewer than all of the portions 848 and/or the vertical struts 840 include the radiopaque markers. For example, every other portion 848, every third portion 848, every fourth portion 848, etc. may include the radiopaque markers. As another example, every third portion 848, every fourth portion 848, etc. may not include the radiopaque markers and the remainder of the portions 848 may include the radiopaque markers. As another example, every other vertical strut 840, every third vertical strut 840, every fourth vertical strut 840, etc. may include the radiopaque markers. As another example, every third vertical strut 840, every fourth vertical strut 840, etc. may not include the radiopaque markers and the remainder of the vertical struts 840 may include the radiopaque markers. In some embodiments, either, both, or any combination of portions 848 and/or the vertical struts 840 may include the radiopaque markers. For example, every other portion 848 may include the radiopaque markers while every other vertical strut 840 off from the every other portion 848 may include the radiopaque markers.

In some embodiments, radiopaque markers may be located on the flange struts 803 and/or the shoulder 845. For example, the flange struts 803 may include a radiopaque material being coined into the distal end of the flange struts 803, such as tantalum, bismuth, iodine, barium, or gold. In some embodiments, the eyelets 806 may be partly or fully filled with the radiopaque markers. Additionally or alternatively, the shoulders 845 may include the radiopaque markers, for example, by partly or fully filling the keyhole opening 830.

In some embodiments, each of the flange struts 803 and/or shoulders 845 may include the radiopaque markers. Additionally or alternatively, fewer than all of the flange struts 803 and/or shoulders 845 include the radiopaque markers. For example, every other flange strut 803, every third flange strut 803, every fourth flange strut 803, etc. may include the radiopaque markers. As another example, every third flange strut 803, every fourth flange strut 803, etc. may not include the radiopaque markers and the remainder of the flange struts 803 may include the radiopaque markers. As another example, every other shoulder 845, every third shoulder 845, every fourth shoulder 845, etc. may include the radiopaque markers. As another example, every third shoulder 845, every fourth shoulder 845, etc. may not include the radiopaque markers and the remainder of the shoulders 845 may include the radiopaque markers. In some embodiments, either, both, or any combination of flange struts 803 and/or shoulders 845 may include the radiopaque markers. For example, every other flange strut 803 may include the radiopaque markers while every other shoulder 845 off from the every other flange strut 803 may include the radiopaque markers.

In some embodiments, one or more of the vertical struts 840 may include the second set of lateral openings 820. The vertical struts 840 may include portions of the frame 802 which may largely remain in the longitudinal direction whether in the expanded or compressed state of the implant 800. For example, as illustrated in FIGS. 9C and/or 9E, when the implant 800 is in the expanded state the vertical struts 840 remain in their original longitudinal orientation, or may include a slight bend or flexion radially outwards towards a distal end 891 of the implant 800. Such radially outward flexion or bend may facilitate formation of the flange (not shown) via the flange struts 803.

In some embodiments, the vertical struts 840 may remain in their longitudinal orientation in the expanded state for at least a majority of the length of the vertical struts 840. In some embodiments, the vertical struts 840 may remain linear in their longitudinal orientation for at least 20%, 30%, 40%, 50%, 60%, 70%, 80%, 90%, or more of the length of the vertical struts 840, e.g., from the shoulder 845 of the implant to a lower arm 843 from which one or more lower cells 849 may form.

In some embodiments, the vertical struts 840 may experience deformation or flexion towards the distal end 891 of the implant 800 at approximately 50% of its length (e.g., halfway between the lower arm 843 and the shoulder 845), at approximately 60% of its length (e.g., 60% of the way from the lower arm 843 to the shoulder 845, or in other words, closer to the distal end 891 of the implant than the proximal end 892 of the implant), at approximately 70% of its length, at approximately 80% of its length, at approximately 90% of its length, and/or at approximately 100% of its length (e.g., at the shoulder 845).

With reference to FIG. 9D, the second set of lateral openings 820 associated with the vertical struts 840 may alternate which edge of the vertical struts 840 from which the lateral openings originate and may extend through a majority or more of a width of the vertical struts 840. For example, as illustrated in FIG. 9D, a first lateral opening 821ba may originate from the right, a second lateral opening 821aa may originate from the left, a third lateral opening 821bb may originate from the right, a fourth lateral opening 821ab may originate from the left, and a fifth lateral opening 821bc may originate from the right. In these and other embodiments, the material 822 of the vertical struts 840 within which the second set of lateral openings 820 are formed may result in a zig-zag shape in the material 822.

In some embodiments, a lateral portion 823 of the material may be narrower than a longitudinal portion 824 of the material 822. In some embodiments, each lateral portion 823 may be the same size as the other lateral portions 823 of the material 822. Additionally or alternatively, the lateral portions 823 may be of varying sizes and/or shapes, examples of which will be described with reference to FIGS. 12A and/or 12B. In some embodiments, variations in the lateral portions 823 and/or the longitudinal portions 824 may determine how far across a width of the vertical struts 840 the second set of lateral openings 820 may extend. In some embodiments, the longitudinal portions 824 may be sized such that the second set of lateral openings 820 extend for at least 40% of the width of the vertical struts 840, at least 50% of the width of the vertical struts 840, at least 60% of the width of the vertical struts 840, at least 70% of the width of the vertical struts 840, at least 80% of the width of the vertical struts 840, and/or at least 90% of the width of the vertical struts 840.

In some embodiments, the shape of the second set of lateral openings 820 may permit flexion of the vertical struts 840 in at least four directions in a plane normal to the longitudinal axis 890 when in the compressed state. For example, the vertical struts 840 may flex to either side as the second set of lateral openings 820 are closed on one edge of the vertical struts 840 and expanded on the opposite side. Continuing the example, for the left side, the lateral openings 821aa, and 821ab may be closed at the left edge and the lateral openings 821ba, 821bb, and 821bc may be opened more widely at the right edge as the vertical struts 840 flexes to the left. Further continuing the example, for the right side, the lateral openings 821ba, 821bb, and 821bc may be closed at the right edge and the lateral openings 811aa and 811ab may be opened more widely at the left edge as the vertical struts 840 flexes to the right. Additionally or alternatively, the vertical struts 840 may flex to the front or the back (e.g., into or out of the page as illustrated in FIGS. 8 and 9D) by slight deformations in the material 822 as the zig-zag shape flexes to the front or the back. Examples of the side-to-side flexion is illustrated in FIGS. 11A-11C and examples of the front to back flexion is illustrated in FIGS. 11D-11F.

In some embodiments, the second set of lateral openings 820 may include an equal number of openings originating on one side as originate on the other side. For example, the second set of lateral openings 820 may include three openings originating at the left and three openings originating at the right. In some embodiments, the second set of lateral openings 820 may include an unequal number of openings originating at the sides, for example, one side may include one more opening than the other side, or more. For example, the second set of lateral openings 820 may include three openings on one side and two on the other, although any number and combination of lateral openings is contemplated. In some embodiments, the number of openings in the second set of lateral openings 820 may be selected based on a tradeoff between material integrity and rigidity compared to the increased flexibility and deformability of the vertical struts 840 as more lateral openings are included.

In some embodiments, the second set of lateral openings 820 may span a relatively short length of the vertical struts 840. In some embodiments, the second set of lateral openings 820 may cover a given span of the overall length of the vertical struts 840 (e.g., from the shoulder 845 to the lower arm 843). In some embodiments, the second set of lateral openings 820 may cover a span of approximately 5%, 10%, 15%, 20%, 30%, 40%, 50%, 60%, 70%, 80%, or 90%, or more of the length of the vertical struts 840.

In some embodiments, all of the vertical struts 840 include the second set of lateral openings 820. In some embodiments, fewer than all of the vertical struts 840 include the second set of lateral openings 820. For example, every other vertical strut 840, every third vertical strut 840, every fourth vertical strut 840, etc. may not include the second set of lateral openings 820. Additionally or alternatively, every other vertical strut 840, every third vertical strut 840, every fourth vertical strut 840, etc. may include the second set of lateral openings 820.

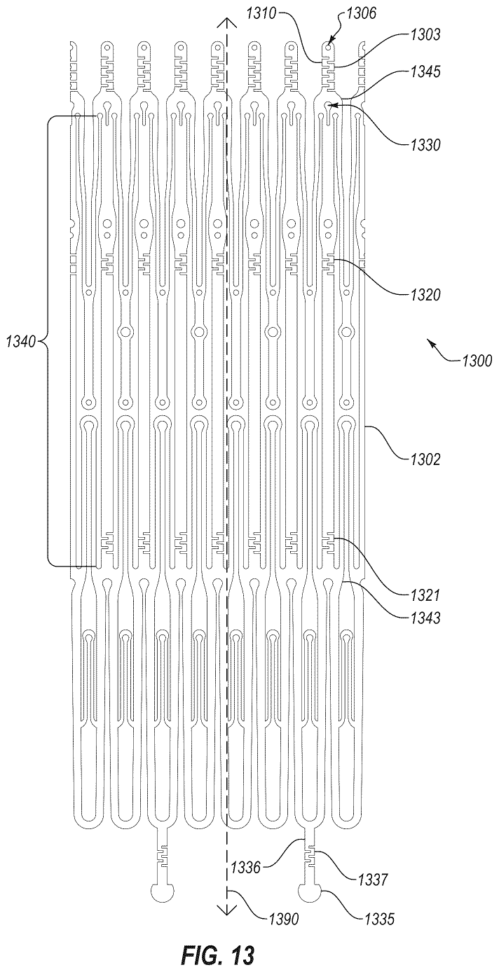

In some embodiments, the vertical struts 840 may include more than one discrete section of the second set of lateral openings 820. One example of such an embodiment may be illustrated with reference to FIG. 13.

In some embodiments, the second set of lateral openings 820 may reduce strain on the vertical struts 840 as they facilitate the mechanical transition from the rigid, straight portions of the vertical struts 840 to the flange struts 803 forming the flange. For example, as seen in FIG. 9E, the second set of lateral openings 820 permits a transition at the second set of lateral openings 820 where the vertical struts 840 change direction to begin to extend radially outward rather than extending parallel to the longitudinal axis 890. In some embodiments, the second set of lateral openings 820 may facilitate the implant 800 accommodating variations in the anatomy of a patient. For example, the anatomy of the patient may have variations in anatomy that causes shifts, jogs, lumps, bumps, or other variations such that an opening within which the implant 800 is to be deployed is not perfectly cylindrical. By providing the second set of lateral openings 820, the implant 800 may better conform to those variations away from a cylindrical shape.

With reference to FIG. 9B, the keyhole opening 830 may include a head portion 832 and a tail portion 834. In some embodiments, the keyhole opening 830 may be disposed in the shoulder 845 to facilitate expansion of the implant 800 from the compressed state to the expanded state. For example, the head portion 832 may provide a void in material in the shoulder 845 that facilitates the flexion of the arms 847a and 847b outwards from the shoulder 845. As another example, by transitioning from the head portion 832 to the tail portion 834, the arms 847a and 847b may have a directed point of flexion in the shoulder 845 (at the head portion 832) while also providing a portion of reinforcement via the more narrow tail portion 834.

By providing the keyhole opening 830, the material stresses in the frame 802 caused by the changes in direction of the frame 802 between the arm 847a, the vertical strut 840, and the arm 847b (such as illustrated in FIG. 9C) when in the expanded state may be alleviated. By alleviating those stresses, the likelihood of material failure at the shoulder 845 may be reduced or mitigated.

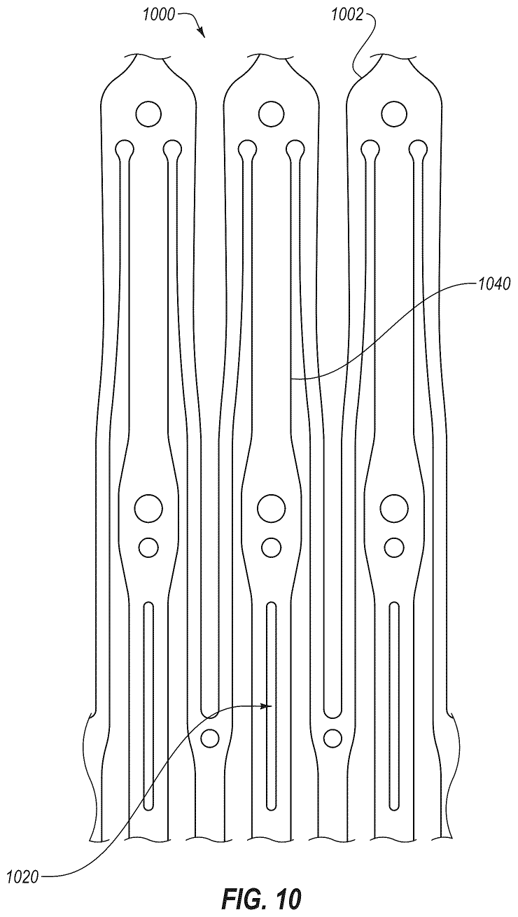

FIG. 10 illustrates a portion of another example implant 1000 configured to dock and/or support one or more prosthetic valves and/or valve components when in a compressed state. The implant 1000 may be comparable or similar to the implant 800 of FIGS. 8-9E. For example, the implant 1000 may include a frame 1002 that may be comparable or similar to the frame 802. The frame 1002 may include vertical struts 1040 that may be similar or comparable to the vertical struts 840 of the frame 802. The frame 1002 may include a slit 1020.

In some embodiments, the slit 1020 may be positioned in the vertical strut 1040 at a similar or comparable location to the second set of lateral openings 820 in the vertical struts 840. The slits 1020 may perform a similar or comparable function for the implant 1000 as the second set of lateral openings 820 for the implant 800. For example, the slit 1020 may reduce strain on the vertical struts 1040 as the vertical struts 1040 transition from the rigid, straight portions of the vertical struts 1040 to extend radially outwards to ultimately form a flange (not shown). For example, the slit 1020 may reduce strain on the frame 1002 at a transition point where the vertical struts 1040 change direction to begin to extend radially outward rather than extending parallel to a longitudinal axis.

FIGS. 11A-11C illustrate an example of flexion of a frame 1102 of an implant 1100 in a side-to-side flexion. FIGS. 11D-11E illustrate an example of flexion of the frame 1102 of the implant 1100 in a front-to-back flexion. The implant 1100 may be comparable or similar to the implant 800 and/or 1000 of FIGS. 8-10. The implant 1100 may include a set of lateral openings 1120, including a first lateral opening 1121a, a second lateral opening 1121b, a third lateral opening 1121c, a fourth lateral opening 1121d, and a fifth lateral opening 1121e. The set of lateral openings 1120 may be comparable or similar to the first set of lateral openings 810 and/or the second set of lateral openings 820. For example, the flexions illustrated in FIGS. 11A-11C and 11D-11E may be representative of the flexion which may occur for the first set of lateral openings 810 and/or the second set of lateral openings 820.

FIG. 11A illustrates a front view of the frame 1102 with the set of lateral openings 1120 without flexion. FIG. 11B illustrates flexion of the frame 1102 of FIG. 11A to the right, and FIG. 11C illustrates flexion of the frame 1102 of FIG. 11A to the left. As illustrated in FIG. 11B, the lateral openings 1121a, 1121c, and 1121e may open more widely at the left edge and the lateral openings 1121b and 1121d may close more narrowly at the right edge as the frame 1102 flexes to the right. As illustrated in FIG. 11C, the lateral openings 1121a, 1121c, and 1121e may close more narrowly at the left edge and the lateral openings 1121b and 1121d may open more widely at the right edge as the frame 1102 flexes to the left.

FIG. 11D illustrates a side view of the frame 1102 of the implant 1100 without flexion. FIG. 11E illustrates flexion of the frame 1102 of FIG. 11D to the left, and FIG. 11F illustrates flexion of the frame 1102 of FIG. 11D to the right.

FIGS. 12A and 12B illustrate additional examples of lateral openings of 1220a and 1220b, respectively, of implants 1200a and 1200b, respectively. FIG. 12A illustrates an embodiment in which the lateral openings 1220a are not evenly distributed in the implant 1200a. FIG. 12B illustrates an embodiment in which the lateral openings 1220b are curved. The implants 1200a and 1200b may be similar or comparable to the implant 800. The lateral openings 1220a and 1220b may be similar or comparable to the first and/or second set of lateral openings 810 and 820.

As illustrated in FIG. 12A, the implant 1200a may include the set of lateral openings 1220a that may include a first lateral opening 1221aa, a second lateral opening 1221ab, a third lateral opening 1221ac, a fourth lateral opening 1221ad, and a fifth lateral opening 1221ae. The implant 1200a may include a frame 1202a that includes a first lateral portion 1223a, a second lateral portion 1223b, a third lateral portion 1223c, and a fourth lateral portion 1223d and a first longitudinal portion 1224a, a second longitudinal portion 1224b, and a third longitudinal portion 1224c.

As illustrated in FIG. 12A, the lateral portions 1223 may progressively become larger. For example, the first lateral portion 1223a may be a first width, the second lateral portion 1223b may be wider than the first lateral portion 1223a, the third lateral portion 1223c may be wider than the second lateral portion 1223b, and the fourth lateral portion 1223d may be wider than the third lateral portion 1223c. More generally any of the lateral portions 1223 may have different widths than any other lateral portions 1223. Additionally or alternatively, the longitudinal portions 1224 may progressively become larger. For example, the first longitudinal portion 1224a may be a first width, the second longitudinal portion 1224b may be wider than the first longitudinal portion 1224a, the third longitudinal portion 1224c may be wider than the second longitudinal portion 1224b, or more generally any of the longitudinal portions 1224 may have different widths than any other longitudinal portions 1224.

In some embodiments, by providing progressively thicker portions of the frame 1202 around the set of lateral openings 1220a, the frame 1202 may be more flexible towards the first lateral opening 1221aa and more rigid towards the fifth lateral opening 1221ae. Doing so may permit the frame 1202 to flex more readily towards a distal end of a component of the frame 1202 while still providing more rigidity towards the proximal end of the frame 1202a. For example, if used for the first set of lateral openings 810 associated with the flange strut 803 of the implant 800, the flange strut 803 may flex less towards the shoulder 845 and flex more towards the distal end of the flange strut 803. As another example, if used for the second set of lateral openings 820 associated with the vertical strut 840 of the implant 800, the vertical struts 840 may flex more towards the shoulder 845 and flex less towards the arm 847.

FIG. 13 illustrates a view of an additional example implant 1300 configured to dock and/or support one or more prosthetic valves and/or valve components. The implant 1300 may be similar or comparable to the implant 800, and includes a similar view to that illustrated in FIG. 8. The implant 1300 may include a frame 1302 with flange struts 1303, vertical struts 1340, shoulders 1345, lower arms 1343, and feet 1335 that may be similar or comparable to the frame 802, the flange struts 803, the vertical struts 840, the shoulders 845, the lower arms 843, and the feet 835 of the implant 800. Additionally, the flange strut 1303 may include a hole 1306 and a first set of lateral openings 1310 that may be similar or comparable to the eyelets 806 and the first set of lateral openings 810. Additionally, the implant 1300 may include and/or utilize one or more radiopaque markers and/or stitching eyelets in a similar or comparable manner to that described with reference to the implant 800.

As illustrated in FIG. 13, the vertical struts 1340 may include a second set of lateral openings 1320 that may be similar or comparable to the second set of lateral openings 820 of the implant 800. The vertical struts 1340 may also include a third set of lateral openings 1321. The third set of lateral openings 1321 may be similar or comparable in configuration, number, and/or size as the second set of lateral openings 1320. Additionally or alternatively, the third set of lateral openings 1321 may include a different configuration, number, and/or size than the second set of lateral openings 1320. By providing the third set of lateral openings 1321, the vertical struts 1340 may be configured to be able to flex, move, or change its form to better accommodate variations in the anatomy of a patient.

In some embodiments, the second set of lateral openings 1320 may be disposed in a distal half of the vertical strut 1340 (e.g., between a midpoint of the vertical strut 1340 and the shoulder 1345) and the third set of lateral openings 1321 may be disposed in a proximal half of the vertical strut 1340 (e.g., between the midpoint of the vertical strut 1340 and the lower arm 1343).

In some embodiments, the frame 1302 may include a fourth set of lateral openings 1337 associated with the foot 1335. For example, the fourth set of lateral openings 1337 may be disposed within a foot strut 1336 extending from a remainder of the frame 1302 to the foot 1335. The fourth set of lateral openings 1337 may be similar or comparable in configuration, number, and/or size as the second set of lateral openings 1320. Additionally or alternatively, the fourth set of lateral openings 1337 may include a different configuration, number, and/or size than the second set of lateral openings 1320. By providing the fourth set of lateral openings 1337, the frame 1302 may be able to flex or move proximate the foot 1335. For example, during deployment of the implant 1300, during repositioning of the implant 1300, and/or during release of the foot 1335, the fourth set of lateral openings 1337 may provide flexibility to the frame 1302 to avoid material failure and/or reduce stress on the implant 1300.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implant comprising: an expandable frame configured to expand from a compressed state to an expanded state when deployed, the expandable frame including one or more vertical struts that each include a span that remains in a longitudinal orientation in both the compressed state and the expanded state, wherein at least one of the vertical struts includes a first set of lateral openings extending partway through the at least one vertical strut.

Example 2. Any of the foregoing examples, such as example 1, wherein the first set of lateral openings alternates which side of the at least one vertical strut at which a respective opening originates between a first side and a second side opposite the first side.

Example 3. Any of the foregoing examples, such as example 2, wherein the first set of lateral openings includes at least two lateral openings on each of the first side and the second side.

Example 4. Any of the foregoing examples, such as example 3, wherein the first set of lateral openings includes three lateral openings on the first side and two lateral openings on the second side.

Example 5. Any of the foregoing examples, such as example 3, wherein the first set of lateral openings includes at least four lateral openings on each of the first side and the second side.

Example 6. Any of the foregoing examples, such as any of examples 1-5, wherein the first set of lateral openings are evenly spaced.

Example 7. Any of the foregoing examples, such as any of examples 1-5, wherein the first set of lateral openings are not evenly spaced.

Example 8. Any of the foregoing examples, such as any of examples 1-7, wherein the first set of lateral openings are normal to the longitudinal orientation.

Example 9. Any of the foregoing examples, such as any of examples 1-8, wherein each of the one or more vertical struts includes a respective first set of lateral openings.

Example 10. Any of the foregoing examples, such as any of examples 1-9, further comprising a second set of lateral openings extending partway through the at least one vertical strut.

Example 11. Any of the foregoing examples, such as example 10, wherein the second set of lateral openings alternates which side of the at least one vertical strut at which a respective opening originates between a first side and a second side opposite the first side.

Example 12. Any of the foregoing examples, such as examples 10-11, wherein the first set of lateral openings are in a first half of the at least one vertical strut and the second set of lateral openings are in a second half of the at least one vertical strut.

Example 13. Any of the foregoing examples, such as example 12, wherein the first set of lateral openings are between a first shoulder at a distal end of the at least one vertical strut and a midpoint of the at least one vertical strut; and wherein the second set of lateral openings are between a second shoulder at a proximal end of the at least one vertical strut and the midpoint of the at least one vertical strut.

Example 14. Any of the foregoing examples, such as any of examples 10-13, wherein the first set of lateral openings and the second set of lateral openings include a same quantity of lateral openings.

Example 15. Any of the foregoing examples, such as any of examples 10-13, wherein the first set of lateral openings and the second set of lateral openings include a different quantity of lateral openings.

Example 16. Any of the foregoing examples, such as any of examples 1-15, further comprising a slit between a first shoulder at a distal end of the at least one vertical strut and a midpoint of the at least one vertical strut.

Example 17. Any of the foregoing examples, such as any of examples 1-16, further comprising an opening in a shoulder at a distal end of the at least one vertical strut.

Example 18. Any of the foregoing examples, such as example 17, wherein the opening includes a keyhole shape with a head portion oriented at the distal end and a tail portion pointing towards a proximal end of the at least one vertical strut.

Example 19. Any of the foregoing examples, such as any of examples 1-18, wherein the expandable frame includes one or more flange struts that, in the expanded state, extend radially away from a distal end of the expandable frame.

Example 20. Any of the foregoing examples, such as example 19, wherein at least one of the one or more flange struts includes a third set of lateral openings extending partway through the at least one flange strut.

Example 21. Any of the foregoing examples, such as example 20, wherein the third set of lateral openings alternates which side of the at least one flange strut at which a respective opening originates between a first side and a second side opposite the first side.

Example 22. Any of the foregoing examples, such as any of examples 19-21, further comprising a skirt coupled to the flange struts to form a flange about the expandable frame.

Example 23. Any of the foregoing examples, such as example 22, wherein the flange extends beyond the vertical struts when the expandable frame is in the expanded state.

Example 24. Any of the foregoing examples, such as any of examples 1-23, wherein the expandable frame includes a foot at a proximal end of the expandable frame and a foot strut connecting the foot to a remainder of the expandable frame.

Example 25. Any of the foregoing examples, such as example 24, wherein the expandable frame includes a fourth set of lateral openings extending partway through the foot strut.

Example 26. Any of the foregoing examples, such as example 25, wherein the fourth set of lateral openings alternates which side of the foot strut at which a respective opening originates between a first side and a second side opposite the first side.

Example 27. Any of the foregoing examples, such as any of examples 1-26, further comprising a valve deployed within a central cavity defined by the expandable frame.

Example 28. Any of the foregoing examples, such as example 27, wherein the valve is configured to be coupled to the expandable frame when in the expanded state.

Example 29. An implant comprising: an expandable frame configured to expand from a compressed state to an expanded state when deployed, the expandable frame including one or more flange struts that, in the expanded state, extend radially away from a distal end of the expandable frame, wherein at least one of the flange struts includes a first set of lateral openings extending partway through the at least one flange strut.

Example 30. Any of the foregoing examples, such as example 29, wherein the first set of lateral openings alternates which side of the at least one flange strut at which a respective opening originates between a first side and a second side opposite the first side.

Example 31. Any of the foregoing examples, such as example 30, wherein the first set of lateral openings includes at least two lateral openings on each of the first side and the second side.

Example 32. Any of the foregoing examples, such as example 31, wherein the first set of lateral openings includes at least four lateral openings on each of the first side and the second side.

Example 33. Any of the foregoing examples, such as any of examples 29-32, wherein the first set of lateral openings are evenly spaced.

Example 34. Any of the foregoing examples, such as any of examples 29-33, wherein the first set of lateral openings are not evenly spaced.

Example 35. Any of the foregoing examples, such as any of examples 29-34, wherein the first set of lateral openings are normal to a longitudinal direction of the implant.

Example 36. Any of the foregoing examples, such as any of examples 29-35, wherein each of the one or more flange struts includes a respective first set of lateral openings.

Example 37. Any of the foregoing examples, such as any of examples 29-36, wherein the expandable frame further includes one or more vertical struts that each include a span that remains in a longitudinal orientation in both the compressed state and the expanded state.

Example 38. Any of the foregoing examples, such as example 37, further comprising a slit between a first shoulder at a distal end of at least one vertical strut and a midpoint of the at least one vertical strut of the one or more vertical struts.

Example 39. Any of the foregoing examples, such as example 38, further comprising an opening in a shoulder at a distal end of at least one vertical strut of the one or more vertical struts.

Example 40. Any of the foregoing examples, such as example 39, wherein the opening includes a keyhole shape with a head portion oriented at the distal end and a tail portion pointing towards a proximal end of the at least one vertical strut.

Example 41. Any of the foregoing examples, such as any of examples 29-40, further comprising a skirt coupled to the one or more flange struts to form a flange about the expandable frame.

Example 42. Any of the foregoing examples, such as any of examples 29-41, further comprising a valve deployed within a central cavity defined by the expandable frame.

Example 43. Any of the foregoing examples, such as example 42, wherein the valve is configured to be coupled to the expandable frame when in the expanded state.

Example 44. A method comprising: extending a delivery capsule through a patient in a first direction to a target location; at least partially deploying an implant at the target location, the at least partially deploying including expanding an expandable frame of the implant thereby causing one or more flange struts of the expandable frame to extend radially away from a distal end of the expandable frame to form a flange, at least one of the flange struts including a first set of lateral openings extending partway through the at least one flange strut and alternating which side of the at least one flange strut at which a respective opening originates between a first side and a second side opposite the first side; and disengaging the implant from the delivery capsule.

Example 45. Any of the foregoing examples, such as example 44, further comprising pulling the implant back in a second direction opposite of the first direction to seat the flange against anatomy of the patient to create a seal between the implant and the anatomy.

Example 46. Any of the foregoing examples, such as example 45, further comprising, after seating the flange against the anatomy, completing any remaining deployment of the implant.

Example 47. Any of the foregoing examples, such as example 46, wherein completing any remaining deployment of the implant comprises releasing a foot of the expandable frame from the delivery capsule and retracting the delivery capsule back through the patient.

Example 48. Any of the foregoing examples, such as any of examples 45-47, wherein the seal is formed by a skirt of material affixed to the implant around the flange and extending at least to a valve seat within the expandable frame.

Example 49. Any of the foregoing examples, such as any of examples 44-48, further comprising installing a valve within the expandable frame at a valve seat within the expandable frame.

Example 50. Any of the foregoing examples, such as example 49, wherein installing the valve comprises: extending a valve delivery capsule with the valve loaded thereon through the patient; deploying the valve within the implant; and fixing the valve to the implant in the valve seat.

Example 51. Any of the foregoing examples, such as examples 44-50, wherein extending the delivery capsule to the target location includes observing radiopaque markers associated with the valve seat in a fluoroscopy image to verify that the implant is at the target location.

Example 52. Any of the foregoing examples, such as any of examples 1-43, further comprising a first set of radiopaque markers located in the vertical struts.

Example 53. Any of the foregoing examples, such as example 52, wherein the first set of radiopaque markers include a radiopaque material coined into an opening in the vertical struts.

Example 54. Any of the foregoing examples, such as any of examples 52-53, wherein the first set of radiopaque markers are located closer to the first set of lateral openings than a shoulder of the frame.

Example 55. Any of the foregoing examples, such as any of examples 52-54, wherein the first set of radiopaque markers include tantalum, bismuth, iodine, barium, or gold.

Example 56. Any of the foregoing examples, such as any of examples 1-43, further comprising a second set of radiopaque markers located in portions of the arms, each of the portions including where two adjacent arms of the frame merge at a terminal end of the arms.

Example 57. Any of the foregoing examples, such as example 56, wherein the second set of radiopaque markers include a radiopaque material coined into an opening in the portions of the arms.

Example 58. Any of the foregoing examples, such as any of examples 56-57, wherein the first set of radiopaque markers include tantalum, bismuth, iodine, barium, or gold.

Example 59. Any of the foregoing examples, such as any of examples 56-58, wherein a respective radiopaque marker of the second set of radiopaque markers is located between where the two adjacent arms merge and the terminal end of the fork.

Example 60. Any of the foregoing examples, such as any of examples 56-59, wherein the radiopaque markers include tantalum, bismuth, iodine, barium, or gold.

Example 61. Any of the foregoing examples, such as any of examples 1-43, further comprising: a first set of radiopaque markers located in the vertical struts; and a second set of radiopaque markers located in portions of the arms, each of the portions including where two adjacent arms of the frame merge at a terminal end of the arms.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope and spirit of these claims.

1. An implant comprising:
an expandable frame configured to expand from a compressed state to an expanded state when deployed, the expandable frame including one or more vertical struts that each include a span that remains in a longitudinal orientation in both the compressed state and the expanded state,
wherein at least one of the vertical struts includes a first set of lateral openings extending partway through the at least one vertical strut.

2. The implant of claim 1, wherein the first set of lateral openings alternates which side of the at least one vertical strut at which a respective opening originates between a first side and a second side opposite the first side.

3. The implant of claim 2, wherein the first set of lateral openings includes at least two lateral openings on each of the first side and the second side.

4. The implant of claim 3, wherein the first set of lateral openings includes three lateral openings on the first side and two lateral openings on the second side.

5. The implant of claim 3, wherein the first set of lateral openings includes at least four lateral openings on each of the first side and the second side.

6. The implant of claim 1, further comprising a second set of lateral openings extending partway through the at least one vertical strut.

7. The implant of claim 6, wherein the second set of lateral openings alternates which side of the at least one vertical strut at which a respective opening originates between a first side and a second side opposite the first side.

8. The implant of claim 1, wherein the first set of lateral openings are in a first half of the at least one vertical strut and the second set of lateral openings are in a second half of the at least one vertical strut.

9. The implant of claim 1, further comprising a valve deployed within a central cavity defined by the expandable frame.

10. An implant comprising:
an expandable frame configured to expand from a compressed state to an expanded state when deployed, the expandable frame including one or more flange struts that, in the expanded state, extend radially away from a distal end of the expandable frame,
wherein at least one of the flange struts includes a first set of lateral openings extending partway through the at least one flange strut.

11. The implant of claim 10, further comprising a skirt coupled to the one or more flange struts to form a flange about the expandable frame.

12. The implant of claim 10, further comprising a valve deployed within a central cavity defined by the expandable frame.

13. The implant of claim 12, wherein the valve is configured to be coupled to the expandable frame when in the expanded state.

14. The implant of claim 10, further comprising a first set of radiopaque markers located in the vertical struts.

15. The implant of claim 14, wherein the first set of radiopaque markers include a radiopaque material coined into an opening in the vertical struts.

16. The implant of claim 14, wherein the first set of radiopaque markers are located closer to the first set of lateral openings than a shoulder of the frame.

17. The implant of claim 14, wherein the first set of radiopaque markers include tantalum, bismuth, iodine, barium, or gold.

18. The implant of claim 14, further comprising a second set of radiopaque markers.

19. A method comprising:
extending a delivery capsule through a patient in a first direction to a target location;
at least partially deploying an implant at the target location, the at least partially deploying including expanding an expandable frame of the implant thereby causing one or more flange struts of the expandable frame to extend radially away from a distal end of the expandable frame to form a flange, at least one of the flange struts including a first set of lateral openings extending partway through the at least one flange strut and alternating which side of the at least one flange strut at which a respective opening originates between a first side and a second side opposite the first side; and
disengaging the implant from the delivery capsule.

20. The method of claim 19, further comprising pulling the implant back in a second direction opposite of the first direction to seat the flange against anatomy of the patient to create a seal between the implant and the anatomy, and, after seating the flange against the anatomy, completing any remaining deployment of the implant.
